(19) United States
(12) Patent Application Publication
(10) Pub. No.: US 2024/0269345 A1
(43) Pub. Date: Aug. 15, 2024

(54) BIODEGRADABLE IMPLANTS WITH ANTICORROSIVE COATINGS CONTAINING SILK FIBROIN

(71) Applicant: University of Georgia Research Foundation, Inc., Athens, GA (US)

(72) Inventors: Hamid Asadi, Athens, GA (US); Ramaraja P. Ramasamy, Athens, GA (US)

(21) Appl. No.: 18/562,997

(22) PCT Filed: May 18, 2022
(86) PCT No.: PCT/US2022/072400

§ 371 Date: Nov. 21, 2023

Related U.S. Application Data
(60) Provisional application No. 63/194,707, filed on May 28, 2021.

(51) Int. Cl. A61L 27/34; A61L 27/04; A61L 27/58; C09D 5/08; C09D 101/02; C09D 189/00

(52) U.S. Cl. CPC A61L 27/34; A61L 27/047; A61L 27/58; C09D 5/08; C09D 101/02; C09D 189/00; A61L 2400/12; A61L 2420/02; A61L 2420/04; A61L 2420/08; A61L 2430/02

(57) ABSTRACT

Biodegradable orthopedic implants and coatings for the implants are provided. The implant can include a Mg alloy and a coating, where the coating includes silk fibroin. The coating can be anticorrosive and can include a first layer comprising polydopamine and a second layer comprising silk fibroin. The silk fibroin can be combined with cellulose nanocrystals. Methods for making coated orthopedic implants are also provided.

### CROSS-REFERENCE TO RELATED APPLICATIONS

This application claims the benefit of and priority to U.S. Provisional Application Ser. No. 63/194,707, having the title “BIODEGRADABLE IMPLANTS WITH ANTICORROSIVE COATINGS CONTAINING SILK FIBROIN”, filed on May 28, 2021, the disclosure of which is incorporated herein by reference in its entirety.

### BACKGROUND

Temporary surgical implants, especially those made from magnesium alloys, are known for their biodegradation and biocompatibility, allowing for an implant that does not require secondary surgery for removal. However, magnesium alloy implants corrode rapidly under physiological conditions, often before bone healing can occur, leading to implant failure.

### SUMMARY

Embodiments of the present disclosure provide biodegradable orthopedic implants, corrosion-resistant coating for orthopedic implants, methods of making implants and coatings, and the like.

An embodiment of the present disclosure includes a biodegradable orthopedic implant. The implant can include a Mg alloy and a coating, where the coating includes silk fibroin.

An embodiment of the present disclosure also includes a corrosion-resistant coating for orthopedic implants. The coating can have a first layer comprising polydopamine and a second layer comprising silk fibroin.

An embodiment of the present disclosure also includes a method of making a coated orthopedic implant in which a magnesium alloy implant can be immersed in an alkaline solution to form an oxide-coated implant. A layer including polydopamine can be disposed on the oxide-coated implant. A coating including silk fibroin can be disposed over the layer comprising polydopamine.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 is a schematic illustration of an example of the fabrication steps of SF-CNC coating on PD-modified AZ31 Mg substrate in accordance with embodiments of the present disclosure.

FIG. 2 is a graph showing article size distribution of CNCs in accordance with embodiments of the present disclosure.

FIGS. 3A-3E are surface SEM images in accordance with embodiments of the present disclosure: AZ31 (FIG. 3A), AZ31-OH (FIG. 3B) AZ31-PD (FIG. 3C), SF (FIG. 3D), and SF-CNC (FIG. 3E); FIG. 3F shows cross-sectional morphology of SF-CNC/substrate interface with the corresponding elemental analysis mapping (FIG. 3G).

FIG. 4 is a graph showing ATR-FTIR spectra of AZ31 (a), AZ31-OH (b) AZ31-PD (c), SF (d), and SF-CNC (e) in accordance with embodiments of the present disclosure.

FIGS. 5A-5C show deconvoluted FTIR spectra of the amide I band of non-treated SF (FIG. 4A), methanol-treated SF (FIG. 4B), and methanol-treated SF-CNC (FIG. 4C); B=β-sheets in accordance with embodiments of the present disclosure.

FIG. 6 is a graph showing water contact angle values of bare, pretreated, and coated Mg samples in accordance with embodiments of the present disclosure.

Photographs of the SF-CNC coating applied on alkaline-treated AZ31 in the absence (A) and presence (B) of intermediary PD layer before and after the cross-cut adhesion test.

FIG. 7 provides camera images of the SF and SF-CNC coatings on alkaline-treated AZ31 with and without the intermediary PD layer before and after the cross-cut adhesion test.

FIGS. 8A-8B show open circuit potential (FIG. 8A) and potentiodynamic polarization (FIG. 8B) curves of bare, pretreated, and coated Mg samples performed in Hank's solution in accordance with embodiments of the present disclosure.

FIGS. 9A-9D provide EIS spectra of Mg samples performed in Hank's solution; Nyquist plots (FIGS. 9A and 9B), Bode-impedance plots (FIG. 9C), and Bode-phase plots (FIG. 9D) in accordance with embodiments of the present disclosure.

FIGS. 10A and 10B provide Nyquist EIS spectra of SF-CNC coating on non-treated (FIG. 10A) and PD-modified (FIG. 10B) AZ31-OH surface and the corresponding cross-sectional SEM images of the coating/substrate interfaces in accordance with embodiments of the present disclosure; Red arrows point to the interfacial gaps between the coating and the substrate.

FIG. 11 shows electrical equivalent circuits employed for fitting the EIS spectra of AZ31 and AZ31-OH (EEC1), AZ31-PD (EEC2), and SF and SF-CNC (EEC3) in accordance with embodiments of the present disclosure.

FIGS. 12A-12C show a sample test setup (FIG. 12A), variation in pH value (FIG. 12B) and hydrogen evolution volume (FIG. 12C) of each group of the samples during immersion in Hank's solution at 37° C. for 14 days.

FIG. 13 provides SEM images of bare, pretreated, and coated Mg samples before and after immersion in Hank's solution for 7 and 14 days at 37° C.

FIG. 14 is a graph showing viability of hFOB cells exposed to sample extracts of 3 and 5 days compared to control cells grown in pure media measured by CCK-8 assay kit in accordance with embodiments of the present disclosure; *** indicates P-value <0.001.

FIGS. 15A-15E provides Fluorescence images of actin-nucleus stained hFOB cells cultured on AZ31 (FIG. 15A), AZ31-OH (FIG. 15B), AZ31-PD (FIG. 15C), SF (FIG. 15D), and SF-CNC (FIG. 15E) for 24 h; Blue: DAPI; Green: F-actin.

FIG. 16 provides a schematic representation of a SF-CNC coating in accordance with embodiments of the present disclosure, along with representative SEM and fluorescence images of the coating.

FIG. 17 is a TEM image of CNC particles in accordance with embodiments of the present disclosure.

FIGS. 18A-18E are surface SEM images (higher magnification) of as-prepared AZ31 (FIG. 18A), AZ31-OH (FIG. 18B) AZ31-PD (FIG. 18C), SF (FIG. 18D), and SF-CNC (FIG. 18E) in accordance with embodiments of the present disclosure.

FIGS. 19A-19C are Nyquist (FIG. 19A), Bode-phase (FIG. 19B), and Bode-impedance (FIG. 19C) plots of bare AZ31 at different time intervals during 7 days of immersion in the Hank's solution in accordance with embodiments of the present disclosure.

FIGS. 20A-20C are Nyquist (FIG. 20A), Bode-phase (FIG. 20B), and Bode-impedance (FIG. 20C) plots of AZ31-OH at different time intervals during 7 days of immersion in the Hank's solution in accordance with embodiments of the present disclosure.

FIGS. 21A-21C are Nyquist (FIG. 21A), Bode-phase (FIG. 21B), and Bode-impedance (FIG. 21C) plots of AZ31-PD at different time intervals during 7 days of immersion in the Hank's solution in accordance with embodiments of the present disclosure.

FIGS. 22A-22C are Nyquist (FIG. 22A), Bode-phase (FIG. 22B), and Bode-impedance (FIG. 22C) plots of SF at different time intervals during 7 days of immersion in the Hank's solution in accordance with embodiments of the present disclosure.

FIGS. 23A-23C are Nyquist (FIG. 23A), Bode-phase (FIG. 23B), and Bode-impedance (FIG. 23C) plots of SF-CNC at different time intervals during 7 days of immersion in the Hank's solution in accordance with embodiments of the present disclosure.

FIG. 24 provides a graph illustrating Variation of log |Z|_{0.01 Hz} values of the samples over 7 days of immersion in Hank's solution. in accordance with embodiments of the present disclosure.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, material science, and the like, which are within the skill of the art.

The following examples are put forth so as to provide those of ordinary skill in the art with a complete disclosure and description of how to perform the methods and use the compositions disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

Abbreviations: SF, silk fibroin; PD, polydopamine; CNC, cellulose nanocrystals; hFOB, human fetal osteoblast cells.

Definitions: As used herein, CNCs or cellulose nanocrystals refer to particles of nanocellulose. Nanocellulose is a term referring to nano-structured cellulose. This may be either cellulose nanocrystal (CNC or NCC), cellulose nanofibers (CNF) also called nanofibrillated cellulose (NFC), or bacterial nanocellulose, which refers to nano-structured cellulose produced by bacteria.

### General Discussion

In accordance with the purpose(s) of the present disclosure, as embodied and broadly described herein, embodiments of the present disclosure, in some aspects, relate to magnesium alloy orthopedic implants including anticorrosive, biocompatible coatings.

In general, embodiments of the present disclosure provide for methods of making the anticorrosive coatings, coating compositions including silk fibroin, and biodegradable implants including the coating compositions.

The present disclosure includes biodegradable orthopedic implants that can include silk fibroin. The implant can contain a magnesium alloy and can biodegrade over time as the surrounding bone heals. Advantageously, the coating can impart anti-corrosive properties to the implant, allowing the implant to biodegrade more slowly. The coating is noncytotoxic.

In some embodiments, the coating can include a first layer and a second layer. The first layer can be an adhesion layer that includes polydopamine, where the first layer is disposed on the implant surface. The second layer can include silk fibroin.

The first layer can include polydopamine. Advantageously, this adhesion layer can assist in adhesion of the second layer. In other embodiments, another equivalent polymer having similar functionality can be substituted for the polydopamine.

In some embodiments, the second layer includes silk fibroin and can also include a biocompatible polymer. The silk fibroin can form a composite with the biocompatible polymer. In other words, CNC is incorporated into the SF matrix. Advantageously, the biocompatible polymer can reinforce the silk fibroin and function as a nanofiller to form a smooth coating layer when compared to an uncoated implant surface. In some embodiments, the biocompatible polymer is cellulose nanocrystal. In some embodiments, the cellulose nanocrystal can be substituted with or used in conjunction with other natural or synthetic polymers with suitable biocompatibility such as lignin as organic nanofiller as well as graphene and its derivatives, hydroxyapatite, ZnO, silica, TiO₂, Mg(OH)₂, halloysite nanotube, and layered double hydroxide as inorganic nanofiller.

In some embodiments, the second layer can include about 0.5-3.0% (w/w) cellulose nanocrystals with respect to silk fibroin weight.

In some embodiments, an external surface of the implant can include a dense oxide layer. While the Mg alloy surface can have a natural oxide layer, in some embodiments the natural oxide layer is polished away and a denser oxide layer with controlled, consistent parameters is formed by an alkaline treatment process. After alkaline treatment, the dense oxide layer is formed on the surface of a Mg alloy to protect the highly susceptible Mg alloy surface against corrosion during the coating procedure. In some embodiments, the oxide layer can be about 4 μm. The dense oxide layer can be formed by immersion of the implant in an alkaline solution. In some embodiments, the alkaline solution is NaOH. The NaOH can have a concentration of about 0.1-5 M, and the immersion time can be about 1-24 hours, depending on the concentration. The coating can be applied to the oxide layer.

The implant can be biodegradable implant, such as a Mg—Al—Zn alloy. In some embodiments, the Mg—Al—Zn alloy can have an Al content of about 3% to 13%, about 2.75% to 5%, or about 3%. In a particular embodiment, the Mg—Al—Zn alloy can be AZ31. In other embodiments, other biodegradable implants can be used, such as porous iron-based implants. In yet other embodiments, non-biodegradable implants can be used such as Ti and its alloy, stainless steel, and Co—Cr implants.

In some embodiments, the oxide layer can be from about 2 μm to 20 μm thick. The first layer can have a sub-micrometer thickness. In some embodiments, the first layer can have a thickness of less than 50 nm. The second layer can be from about 8 μm to 15 μm, or about 8.7 μm to 13.7 μm thick.

Described herein is an anti-corrosive polymeric coating as above, which includes a biocompatible polymer and silk fibroin. The coating can be included on temporary orthopedic implants, such as implants comprising magnesium alloys.

Described herein is a coating material based on silk fibroin (SF) and cellulose nanocrystals (CNCs). The coating material can be applied to an alloy (e.g., a biodegradable AZ31 Mg alloy). Advantageously the coating material provides corrosion resistance and biocompatibility. In a particular embodiment, before the application of the coating, the AZ31 substrate can be modified with a layer of polydopamine to enhance the adhesion of the protective coating to the metal surface. In some embodiments the coating material can have a thickness of 11.2±2.5 μm when applied to the alloy. The coating thickness can be varied by changing the volume of drop casted polymer solution or the number of deposited layers on the metal surface. The coating thickness can be about 5 μm to 50 μm using the drop-cast method.

In some embodiments, additional layers can be added. For example, additional layers of polydopamine can be alternated with SF or SF-CNC coating, such that the outermost layer is SF or SF-CNC. Alternatively, additional coatings of SF or SF-CNC can be added on the second layer to provide a thicker coating.

Embodiments of the present disclosure also provide for methods of making coated orthopedic implants as above. The method can include immersing a magnesium alloy implant in an alkaline solution to form an oxide-coated implant and coating the oxide-coated implant with a silk fibroin solution or a silk fibroin-cellulose nanocrystal solution. A layer including polydopamine can be included between the oxide layer and the silk fibroin-containing layer.

Advantageously, the coating described herein can prevent, delay and/or mitigate formation of hydrogen-forming gas pockets around the implant.

The implants described herein can include but are not limited to such as a screw, a plate, a nail, a pin, a rod, or a prosthesis, as can be envisioned by one of ordinary skill in the art.

### Examples

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

Magnesium (Mg)-based alloys have shown great potential as biodegradable orthopedic implants during recent years. In addition to their excellent biocompatibility and mechanical properties suitable to natural human bones, the ability of Mg alloys to degrade into non-toxic and bioresorbable products makes them highly desirable materials for temporary bone implants applications. Despite their numerous advantages over the commonly used implant materials, the wide clinical application of Mg alloys is still limited mainly because of their susceptibility to corrosion and rapid degradation [1].

Surface modification with polymeric coatings has been widely studied as a feasible approach to mitigate the corrosion of Mg alloys [2-4]. Apart from enhancing corrosion resistance, the polymeric coatings may provide other functionalities to the underlying substrate such as enhanced biocompatibility and cellular responses. Over the past few years, a variety of natural and synthetic polymers have been used for fabrication of corrosion protective coatings on biomedical Mg alloys [5]. Predominantly, synthetic polymers such polylactic acid (PLA), poly(lactic-co-glycolic) acid (PLGA), polyethylene imine (PEI), and polycaprolactone (PCL) have been proven to offer good anticorrosion properties, however, the cellular responses toward these coatings are not often as satisfactory mainly because of their hydrophobic characteristics and lack of bioactivity [6, 7]. On the other hand, natural polymer-based coatings possess enhanced biocompatibility (e.g. conductive to enhanced cell attachment and growth) compared to synthetic polymer-based coatings. However, most biopolymer-based coatings are highly susceptible to the permeation of water and other corrosive species in the physiological environment because of their porous structure and limited barrier properties [8]. In order to take advantage of the desired biocompatibility properties of biopolymers for application as protective coatings on Mg-based implants, their corrosion resistance must be improved in order to be competitive against synthetic polymers.

Several approaches have been attempted to ameliorate the corrosion resistance properties of polymeric coatings on Mg alloys including the incorporation of corrosion inhibitors/organic salts [9, 10], hybridization with other polymers (copolymerization, blending, and layer-by-layer) [11, 12], surface pretreatment/activation [13, 14], and applying intermediate layers [15, 16]. Introduction of anticorrosive nanofillers into polymeric matrix is another well-established approach to enhance the corrosion resistance and durability of polymeric coatings. Addition of anticorrosive nanofillers can improve the barrier properties of polymeric coatings against the aggressive physiological environment by increasing the tortuosity of corrosive species permeation pathways, creation of microcapillaries with high capillary pressure for penetration of liquid species, as well as other mechanisms related to filler-matrix and filler-metal interface interactions [17]. A variety of anticorrosive nanofillers such as graphene and its derivatives [18], hydroxyapatite [19], ZnO [20], silica [21], TiO₂ [22], Mg(OH)₂ [23], and layered double hydroxide [24] have been incorporated into protective polymeric coatings on Mg alloys in previous reports.

Over the past decade, bio-based nanofillers have gained extensive attention due to their low-price, renewability, biocompatibility, and biodegradability [25]. Cellulose nanocrystal (CNC) is a needle-like nanoparticle with the size of 5-50 nm in diameter and hundreds of nanometers in length, which are produced from various plant sources through strong acid hydrolysis [26]. Having high aspect ratio, biocompatibility, excellent mechanical properties, dispersibility in aqueous and organic solvents, and formation of strong interfacial bonding with polymeric matrices through its abundant surface hydroxyl groups are some properties that have made CNC a popular reinforcing agent for fabrication of polymeric composites in various biomedical applications [26]. Recently, the anticorrosion properties of CNC have gained interest and have been reported in a few studies [27-31]. Taking all the desirable properties of CNC into account, we hypothesized it could be a novel and renewable reinforcement material for fabrication of anticorrosive coatings on Mg biodegradable implants.

This study investigates a composite coating consisting of silk fibroin (SF) and CNC on the AZ31 Mg alloy to improve its corrosion resistance and biocompatibility for biodegradable bone implant applications. SF is a natural protein extracted from Bombyx mori silkworm [32]. Owing to its excellent biocompatibility, controllable biodegradability, and superior mechanical properties, SF films and coatings have been extensively researched in diverse biomedical applications [33]. Specifically, SF has been used as surface coating on bone implants in several studies and showed high osteogenic activities both in vitro and in vivo [34]. Compared to the commonly used synthetic polymeric coatings such as polyhydroxybutyrate (PHB), PCL, polyamide, PLA, and PGA, which degrade into acidic products, SF shows less immunogenicity (the human body's immune response provoked by a foreign material) as it decomposes to its constituent biocompatible amino acids [35]. Considering all the above advantages, SF shows great potential as a coating material for biodegradable Mg alloys.

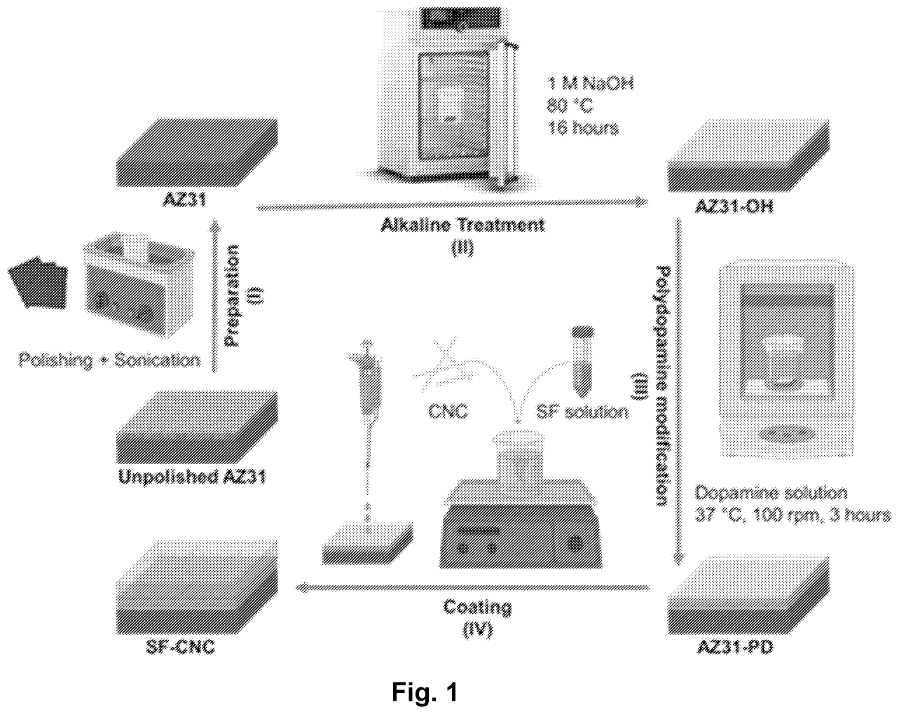

In this study, CNC nanoparticles were incorporated into the SF coating as anticorrosion nanofillers for the first time in reported literature with the aim of improving the barrier and corrosion protection properties of the SF film. A schematic of the coating is shown in FIG. 1. AZ31 was selected as a model biodegradable Mg substrate due to its lower content of Al (˜3 wt %), an element known to be harmful for neural system and bone cells, among all other Mg alloys with typical Al content of 3-13 wt % [36]. Adequate interfacial adhesion is a primary requirement for any protective coating, which is often difficult to achieve on bare metallic surfaces with no functional groups and poor adhesiveness. To address this issue, before applying the polymeric coating, the AZ31 substrates were modified with a thin layer of polydopamine (PD) to serve as intermediary layer and to improve the coating/substrate adhesion. PD possesses outstanding adhesiveness to almost any type of material, numerous functional groups, and excellent biocompatible, which make it a very suitable platform for modification of biomedical Mg alloys surfaces before applying a secondary protective coating [37]. Finally, the composite CNC-reinforced SF coating was applied onto the PD-pretreated AZ31 surface by the drop-cast method. The proposed composite coating was characterized through various physiochemical techniques. The corrosion resistance of the coating and the effect of CNC inclusion on the corrosion protection performance of the coating were investigated through extensive electrochemical techniques and in vitro immersion tests. Finally, the biocompatibility of the coating in terms of cellular toxicity and adhesion was assessed.

The main advantages of this combination are that it can improve the anti-corrosion properties in two ways. First, the CNC has anticorrosive properties, which can be imparted to any other polymeric systems, as long as a composite can be formed between them. Second, the interaction between SF and CNC changes the secondary structure of SF (increased 3-sheet content) and boosts the anti-corrosion and barrier properties of the SF coating. This synergistic combination results in increased performance expected from the individual components.

### Materials and Methods

Materials—Bombyx mori silk cocoons were obtained from Paradise Fibers (USA). Spectra/Por 3 regenerated cellulose dialysis membrane tubing (3.5 kDa, molecular weight cut-off) was obtained from Fisher Scientific (USA). Concentrated aqueous suspension of CNC was kindly provided by Dr. Sudhagar Mani, University of Georgia, USA. Polyethylene glycol with a molecular weight of 200 (PEG₂₀₀) was purchased from Sigma-Aldrich (Cat #P3015). Trypsin-EDTA was obtained from Corning (Manassas, VA 20109). The Cell Counting Kit-8 (CCK-8) was purchased from Sigma-Aldrich (St. Louis, MO 63103). Mixture of Dulbecco's Modified Eagle's Medium and Ham's F-12 Medium (DMEM/F-12), geneticin, and fetal bovine serum (FBS) were bought from Gibco-Life Technologies (Grand Island, NY 14072). The phalloidin (Alexa Fluor 488) and DAPI (diamidino-2-phenylindole) reagents were purchased from Thermo Fisher Scientific (Thermo Fisher Scientific Inc., USA). Fetal Osteoblast cells were purchased from American Type Culture Collection (hFOB 1.19-ATCC 11372). All aqueous solutions were prepared using ultrapure water purified with a Milli-Q Millipore system. All other reagents, unless otherwise specified, are of reagent grade.

Silk Fibroin Extraction—Aqueous SF solution was extracted from Bombyx mori cocoons following a previously established procedure [38]. The cocoons were first boiled in a 0.02 M sodium carbonate solution for 30 min to remove the sericin. After drying the degummed SF at room temperature overnight, it was dissolved in 9.3 M lithium bromide solution for 4 h at 60° C. Then, the SF solution in lithium bromide was dialyzed against water for 48 h to obtain the aqueous SF solution which was further filtered and stored at 4° C. until used. The final concentration of SF solution was 7% w/v.

Preparation of AZ31 Substrates—AZ31 Mg alloy sheets ((wt %): Mg (balance), Al (2.5-3.5), Zn (0.7-1.3), Mn (0.2-1), Si (0.5), Cu (0.01), Fe (>0.5), Ni (>0.5), others (0.4)) with dimension of 2×2 cm and thickness of 1 mm were polished by 2000, 2500, and 3000-grit SiC abrasive papers to remove the natural metal oxides layer and obtain a uniform roughness. Then, they were sonicated in absolute ethanol and water each for 5 min to remove any surface contaminations, and finally dried at 60° C.

Pretreatment of AZ31 Substrates—AZ31 samples were treated with 1 M NaOH at 80° C. for 16 h to form a passive layer of Mg(OH)₂ on the Mg alloy surface. Afterward, they were thoroughly rinsed with water, dried at 80° C. for 1 h, and labeled as “AZ31-OH”. The alkaline treatment of AZ31 substrates promotes the deposition of a homogenous PD layer by mitigating the generation and adhesion of H₂ to the Mg surface [39]. The PD modified samples were obtained by placing the alkaline treated Mg substrates in a 6-well plate filled with 10 mL of 2 mg/mL dopamine hydrochloride solution in Tris-HCl buffer (25 mM, pH 8.5). The reaction was carried out in a shaker incubator 37° C. and 100 rpm for 3 h at dark. After completion of the reaction, the samples were sonicated with water for 1 min, and labelled as “AZ31-PD”

Fabrication of the SF and SF-CNC coatings—SF-based protective coatings on the Mg alloy were prepared by the solution casting method from aqueous SF and CNC solutions. First, CNC dispersion with a concentration of 7 mg/mL was obtained by adding a certain amount of water to the stock CNC, followed by stirring and bath sonication each for 15 min. The obtained solution was filtered (5 μm) to remove the big particles from the solution. In this study, we employed never-dried aqueous dispersions of CNC over its lyophilized powder form to reduce the chance of CNC aggregation, which is known to be a common issue in producing CNC-reinforced polymeric composites [40]. A certain volume of CNC suspension was added to the SF solution and stirred for 15 min to obtain a well-dispersed and homogenous solution. To obtain better mechanical properties and prevent surface crack formation on the coatings, PEG200 was also added into the solution. Finally, the pH of the solution was brought up to 10 using a 1 M NaOH solution. The detailed composition of the coating solutions is listed in Table 1. To prepare the top coatings, 200 μL of either SF or SF-CNC solution was pipetted on the pretreated AZ31 substrates and dried at room temperature for 24 h. Afterward, the SF-based coatings were cross-linked by immersion in 80 wt % methanol for 1 h to become more water stable and corrosion resistant, as reported previously [41]. The coated samples with the blank SF and CNC-containing solution were denoted as “SF” and “SF-CNC”, respectively. A batch of SF-CNC coatings were also made on AZ31-OH substrates without PD modification under the same condition to be used as control samples in adhesion tape and electrochemical corrosion tests.

TABLE 1
Compositions of the solutions
used for the fabrication of coatings.

| | 7% w/v | 7 mg/mL CNC | PEG₂₀₀ | 1M NaOH | Water |
|---|---|---|---|---|---|
| Solutions | SF (μL) | dispersion (μL) | (μL) | (μL) | (μL) |
| SF | 1000 | 0 | 10 | 10 | 100 |
| SF-CNC | 1000 | 100 | 10 | 10 | 0 |

Material Characterization— Dynamic light scattering (DLS; Zetasizer, Malvern Instrument) was performed to measure the particle size and surface charge (zeta potential) of CNC particles. Dilute CNC dispersions (0.1 mg/mL in water) were used to avoid multiple scattering [42]. The surface and cross-sectional morphologies of the samples were observed using a field emission scanning electron microscope (SEM, FEl Teneo, FEl Co.). The elemental analysis across the coating/substrate interface was obtained by an energy dispersive x-ray spectrometer (EDS) connected to the SEM. The chemical structures of the coatings were characterized by attenuated total reflectance-Fourier transform Infrared (ATR-FTIR) analysis using a Nicolet 6700 spectrometer (Thermo Electron Corporation, MA, USA). The spectra were recorded over the wavenumber range of 4000-800 cm-1 with 128 scans and a resolution of 1 cm-1 for each measurement. Amid I band deconvolution was performed for each spectrum to measure the crystallinity (β-sheet content) using OriginPro 8 software according to previous studies [43]. Water contact angle (WCA) measurements were performed using a Kruss DSA 100 drop shape analyzer at room temperature. The static contact angle was measured by the dropwise addition of water (1 μL) onto 2×2 cm samples. Measurements were taken at 3 different areas for each sample. Adhesion strength of the coatings were evaluated by ASTM D3359 cross-cut tape test. Two series of 11 cuts with 1 mm distance apart were made perpendicular to each other using a sharp blade and a cross-guide to form a lattice pattern of 100 squares on the coatings. An adhesive tape (SEMicro CHT) was applied to the cross-cut area, rubbed with an eraser to ensure a firm contact between the tape and the test area, and quickly pulled off at an angle of 1800 after 90 s. The percentage of the coating detached by the tape was quantified and the coating adhesion was graded according to ASTM standard chart, with 5B indicative of the highest level of adhesion (0% detachment) and OB indicative of the worst adhesin (>65% detachment).

### In Vitro Corrosion Evaluation

Electrochemical Corrosion Measurements—All the electrochemical and in vitro corrosion studies were carried out in Hank's solution (NaCl 8.00 g/L, KCl 0.40 g/L, CaCl₂ 0.14 g/L, NaHCO₃ 0.35 g/L, MgSO₄·7H₂O 0.10 g/L, MgCl₂·6H₂O 0.1 g/L, Na₂HPO₄·2H₂O 0.6 g/L, KH₂PO₄ 0.6 g/L, D-Glucose 1 g/L, NaHCO₃ 0.35 g/L, pH 7.4) in order to simulate the corrosive physiological environment according to ASTM-G31-72 standards [8].

Electrochemical corrosion tests were performed in a custom-made three-electrode cell consisting of Mg samples with an exposing surface area of 1 cm2 served as working electrode, a silver/silver chloride (Ag/AgCl 3 M) reference electrode, and a platinum wire as the counter electrode connected to an electrochemical workstation (CHI-920c, CH Instruments Inc., Austin, TX). The open circuit potential (OCP) plot versus time were recorded over a course of 3 h under no external potential. Potentiodynamic polarization (PP) test was performed from −200 mV to +200 mV vs OCP at a scan rate of 1 mV/s and the values of corrosion potential (E_{corr}) and corrosion current density (I_{corr}) for each sample were calculated using the Tafel extrapolation method. For electrochemical impedance measurement (EIS), the samples were allowed to stabilize for 30 min in the electrolyte before the test. Then, EIS measurement was run at the OCP over a scanning frequency range of 105 to 10⁻² Hz with an AC amplitude of ±10 mV. All EIS data were fitted and analyzed using Zview software by adopting appropriate electrical equivalent circuit (EEC) models. For each of OCP, PP, and EIS studies, a new batch of samples were used, all in triplicates, and the average result was reported and shown in the figures.

In vitro Corrosion Tests—Each sample was individually placed in a sealed test containing Hank's solution with a volume to sample area ratio of 20 mL/cm² (ASTM G31-72). An inverted funnel-burette system was placed above the samples inside the solution to collect the generated hydrogen every day up to 14 days. Meanwhile, the pH value of the corrosion media was also measured at the same time intervals. At the end of the immersion test, the specimens were removed from the solution, rinsed thoroughly with water, and dried at 70° C. The post-corrosion morphologies of the samples were imaged by SEM.

### Cytocompatibility

Cell Culture—Fetal Osteoblast cells were cultured in 75 cm² T-flasks with a 1:1 mixture of Ham's F12 Medium and Dulbecco's Modified Eagle's Medium, containing 0.3 mg/mL Geneticin and supplemented by 10% fetal bovine serum (FBS) in a humidified incubator at 37° C. with 5% CO₂. The culture medium was refreshed every day until cells reached 90% confluency. Thereafter, the cells were detached from the T-flask surface by treating them with 0.18% trypsin and 5 mM EDTA for 5 min.

Cell Viability—The samples leachates were obtained using cell culture media with an extraction media/surface area ratio of 1.25 mL/cm² in a humidified atmosphere with 5% CO₂ at 37° C. for 3 and 5 days according to ISO 10993-12 [44]. The leachates were then exposed to hFOB cells to evaluate any potential cytotoxic response. Cells were seeded in a 96-well plate at a concentration of 50,000 cells/mL and incubated for 24 h in a humidified incubator with 5% CO₂. After formation of a cell monolayer on the bottom of the wells, the medium in each well was replaced with 100 μL of the sample leachates (n=6) and incubated for another 24 h. The cytotoxicity assay was performed based on the manufacturer's protocol (Sigma-Aldrich) using a CCK-8 kit. After 24 h of cells exposure to the leachates, 10 μL of WST-8 solution was added to each well and incubated for 4 h. Afterwards the UV absorbance of the orange formazan produced by the live cells was measured at 450 nm and the relative cell viability was calculated according to the following equation [45]:

$$\% cell viability = \frac{absorbance of the test samples}{absorbance of the control samples} \times 100$$

Cell Adhesion—Samples with a size of 1×1 cm were sterilized by UV radiation for 2 h on each side prior to cell seeding. Osteoblast cells at a concentration of 15,000 cells/cm² were seeded on each sterilized sample and kept for 24 h in an incubator (T=37° C., 5% CO₂). The cell-seeded samples were washed with a PBS solution, fixed with 4% formaldehyde for 10 min, and permeated with 0.2% Triton X-100 for 10 min. After several washing with PBS, the cells cytoplasm and nuclei were stained using phalloidin and DAPI reagents, respectively, to observe their morphology and adhesion on different samples. An inverted fluorescent microscope (AMG EVOS FL) was used to image the stained cells.

Statistical analysis—All data in this study were expressed as mean±standard deviation with n=3. One-way analysis of variance (ANOVA) was adopted for statistical analysis, where P-value <0.5 was considered statistically significant.

### Results

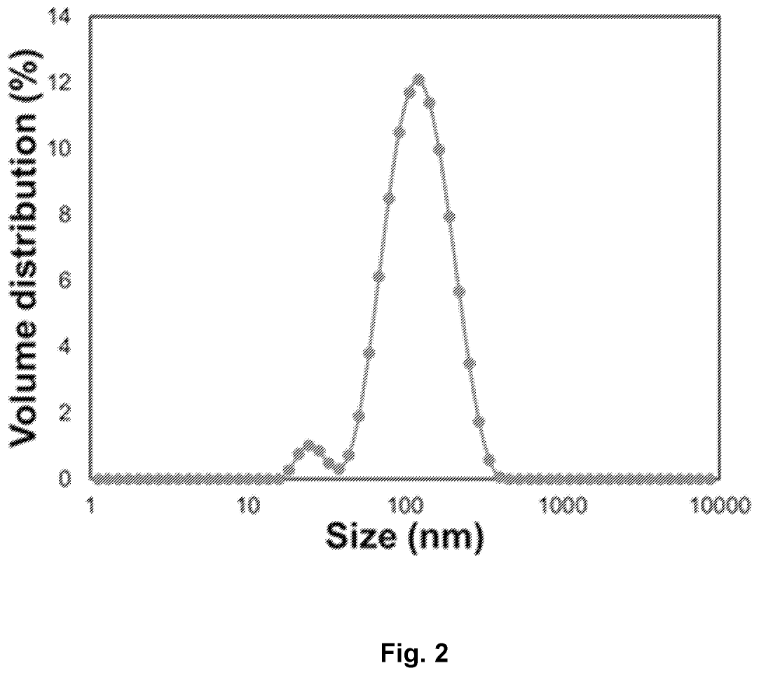

CNC Characterization— The CNC dispersion was analyzed by DLS (FIG. 2). The rod-like morphology of CNCs resulted in emergence of two peaks at 132 and 26 nm, where the smaller one represents the diameter and the larger one represents the length of the CNCs. The sizes were comparable with the previous reports [46]. Besides, the zeta-potential of CNCs were found to be −31.4±2.1 mV, implying the anionic surface of CNC particles [31].

### Coating Characterization

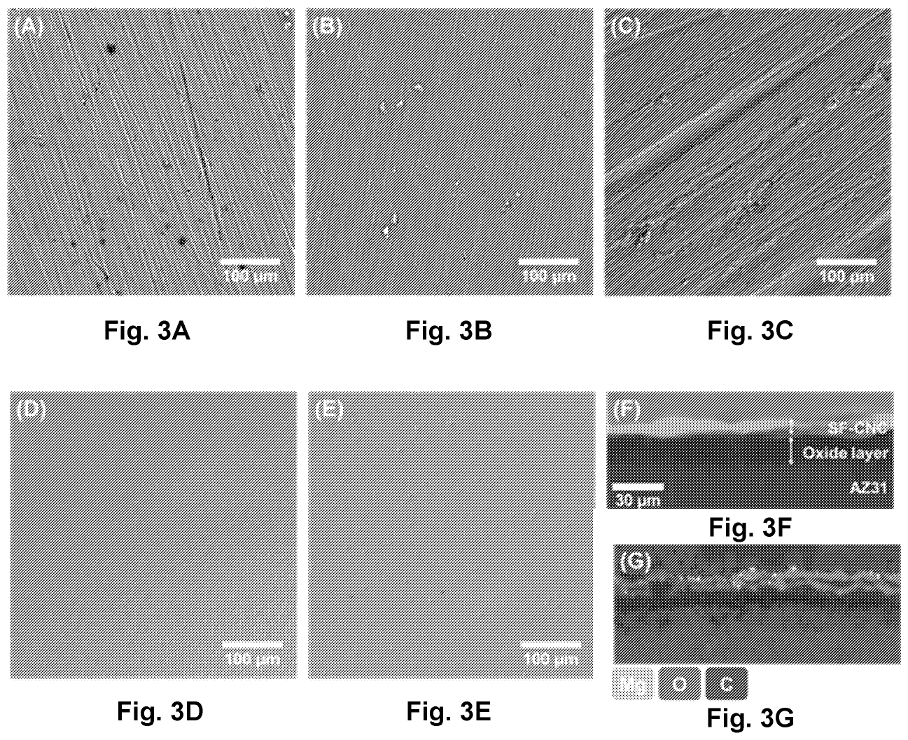

Surface Morphology—The surface morphologies of Mg substrates before and after modifications were investigated by SEM as shown in FIGS. 3A-3C. Untreated AZ31 substrate showed a quite flat surface with some aligned scratches remained form the mechanical polishing step with SiC papers. After alkaline treatment with NaOH, those aligned scratches faded from the surface due to the formation of a dense Mg(OH)₂ oxide layer. AZ31-PD exhibited a rougher surface with some small PD aggregations formed on the surface by self-assembly during the deposition process. Nevertheless, the coating was still aligned with the polishing grooves underneath, implying the relatively low thickness of the PD coating [37]. The surface morphology of the pure SF coating was smooth and homogenous with no cracks or defects on the surface. Being defect-free is a crucial factor for any protective coating, as it ensures the complete isolation of the covered metal against corrosive environment. Similarly, the SF-CNC coating was flat and uniform with no apparent big size CNC agglomeration on the surface, showing that the CNCs were homogeneously distributed within the SF coating. In addition, the cross-sectional SEM image of the SF-CNC coated sample (FIG. 3F) displayed a distinct and compact polymeric layer covering the underlying AZ31 substrate with no void or cleavage at the interface, along with an oxide layer formed on the surface during alkaline treatment. The thicknesses of the SF-CNC coating and the oxide layer were measured to be 11.2±2.5 μm and 20.3±1.8 μm, respectively. The results of EDS analysis (FIG. 3G) further confirmed the elemental composition of each layer, where the highest concentrations of Mg, O, and C elements were observed withing the AZ31 substrate, inner oxide layer, and the outer organic layer composed of SF-CNC coating and PD layer, respectively.

The results of the electrochemical corrosion and in vitro immersion tests clearly demonstrated an enhanced corrosion resistance of the SF coating after the incorporation of CNCs. Compared to the unmodified Mg alloy, the SF-CNC coated AZ31 exhibited a remarkably improved cytocompatibility with a viability of 114% and excellent adhesion and spreading of human fetal osteoblast cells onto the coating surface. The findings of this work highlight the great potential of SF and CNC as bio-based nature-derived anticorrosive nanofillers for fabrication of protective and biocompatible coatings on Mg-based biodegradable orthopedic implants.

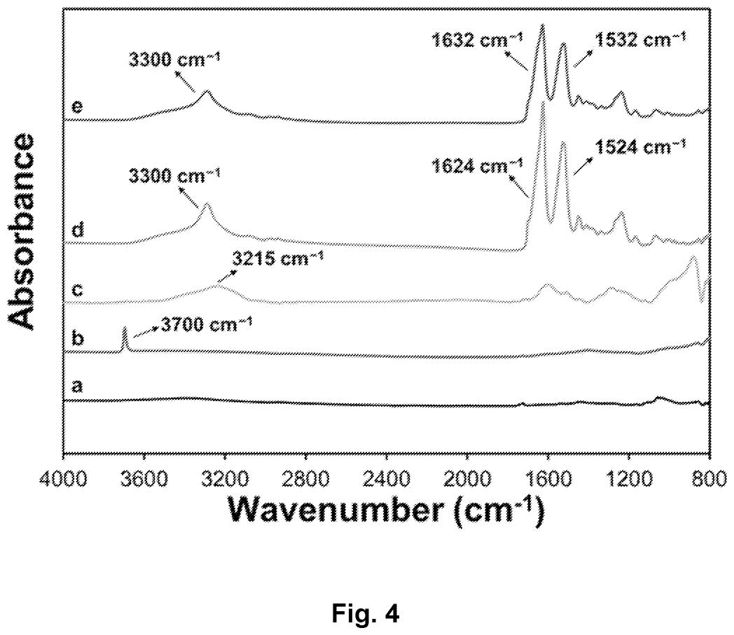

Chemical Composition of the Coatings—FIG. 4 displays the ATR-FTIR spectra of the bare and modified Mg alloy samples. No distinctive peaks were observed in the spectrum of AZ31, as there is no functional group on the surface of bare AZ31 alloy. AZ31-OH showed an adsorption peak at 3700 cm⁻¹, which is related to the hydroxyl groups of the formed Mg(OH)₂ layer during alkaline treatment process [47]. The formation of intermediary PD layer was confirmed by the emergence of the adsorption peaks in the range of 1450-1625 cm⁻¹ corresponding to the C═C stretching vibration of benzene rings and the N—H bending [48], as well as a broad band centered at 3215 cm⁻¹ related to stretching vibrations of —NH (catechol groups) and —OH (hydrogen bonding of PD molecules) groups [49]. The SF coating showed the typical characteristic peaks of SF at 1624 cm⁻¹ and 1524 cm⁻¹, corresponding to the amide I (N—H deformation) and amide II (C—N stretching), respectively. Meanwhile, a broad peak centered at 3300 cm⁻¹ was also observed, which could be attributed to the hydroxyl groups [50]. The SF-CNC coating exhibited a similar spectrum as the SF coating, with some variations. Compared to the pure SF coating, the characteristic bands of SF-CNC shifted to higher wavenumbers and became less pronounced. These alterations suggested the formation of strong intermolecular hydrogen bonding between the SF matrix and CNCs [51, 52].

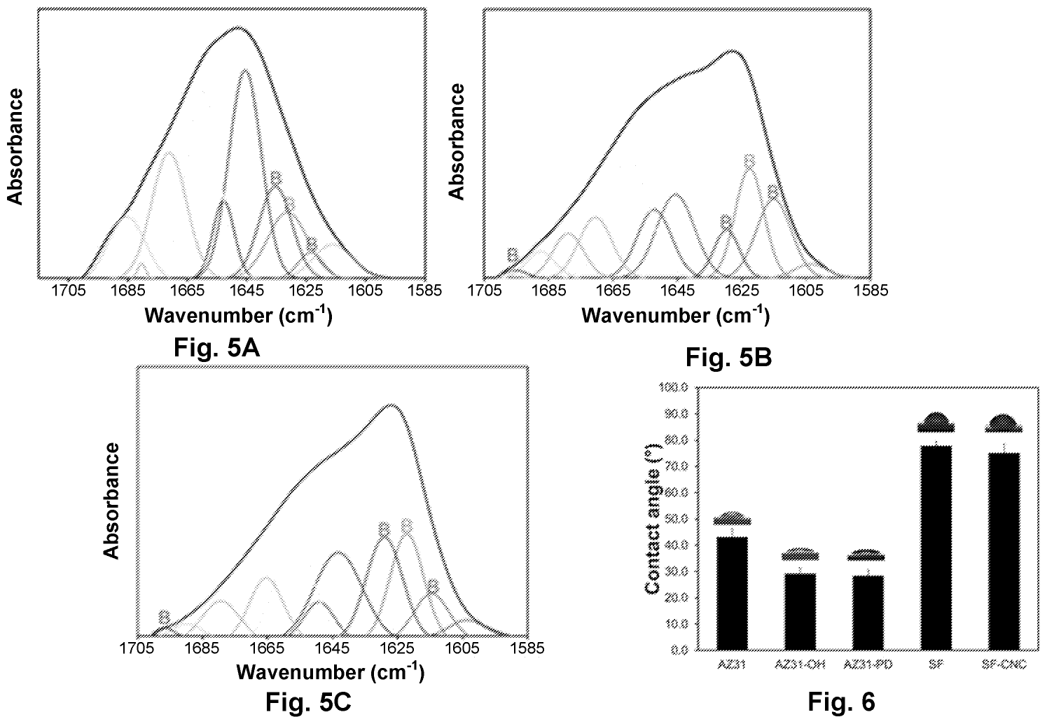

SF has various secondary structures including random coils, α-helices, β-sheets, and β-turns that can be adjusted by change in solvent, pH, applied shearing force, metal ion content as well as post-treatment. The secondary structure determines some of the characteristics of SF such as stability, wettability, mechanical properties, and biodegradability, which are among the most important properties in coating applications [10]. The effect of methanol treatment and CNC incorporation on the secondary structures of the SF coating was quantitively studied by deconvolution of amide I band (1700-1590 cm⁻¹), as shown in FIGS. 5A-5C. As expected, the methanol treatment of SF coatings led to a significant increase in β-sheet percentage from 16% to 23%. Moreover, incorporation of CNCs into the SF coatings was found to further increase the β-sheet content to 31% in the SF-CNC coating. A similar increasing trend in 3-sheets content by nanocellulose has been previously reported and attributed to the strong interaction between CNCs and SF through hydrogen bonding, leading to conformational changes of SF from random coil/helix to β-sheet [53, 54]. The higher content of β-sheet has been reported to be more preferable in protective coating applications, as it improves the water stability and barrier properties of the SF coatings, resulting in higher corrosion protection properties in the physiological environment [55]. The present disclosure is the first time that CNC has been used as an anticorrosive nanofiller for implant application. Although CNC's have been used previously in implant applications, they have been only envisioned as mechanical reinforcement agents, bioactive agents, etc., but not as an anticorrosive nanofiller.

Wettability Measurement—FIG. 6 displays the measured water contact angle (WCA) values of the samples and their representative images. Bare AZ31 substrate showed a relatively hydrophilic surface with an average contact angle of 42.9±3.0°. The contact angle value of AZ31 substrates dropped to 29.1±2.3° after alkaline treatment due to the emergence of numerous hydroxyl groups on the AZ31 surface during alkaline treatment process [56]. No significant change in WCA values was observed following the modification of AZ31-OH surface with PD. Meanwhile, the SF-coated Mg sample showed an apparently less hydrophilic surface with a contact angle value of 77.7±1.6°, which is in agreement with the reported values for methanol-treated SF coatings in previous studies [57]. Incorporation of CNCs into the SF coating did not bring about a significant change in WCA value. Despite the presence of numerous hydrophilic hydroxyl and sulfate groups on the surface of CNCs [31], the higher β-sheet content of the SF-CNC coating reduced hydrophilicity and counterbalances the effect of CNC addition, thus resulted in no significant change (P-value >0.05) in the WCA value [58]. Taking the results of SEM morphological analysis, ATR-FTIR and wettability measurements into consideration, it can be concluded that all the surface modification and coating processes were successfully carried out on the AZ31 alloy substrates.

### Coating Adhesion Test—

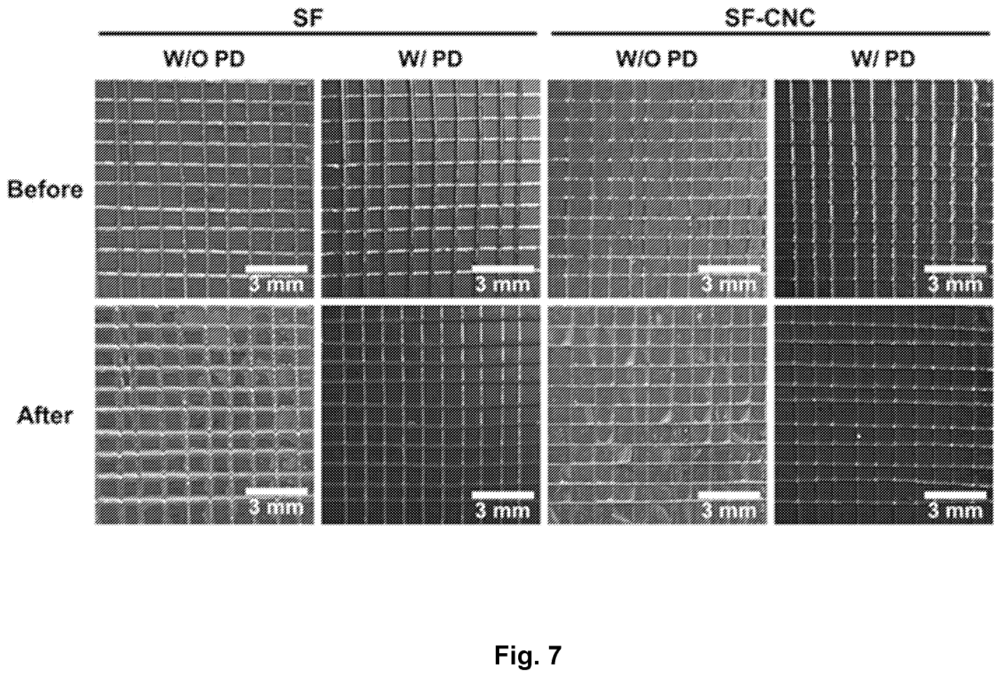

The performance and reliability of any protective coating highly relies on the adhesion strength of the coating to the underlying substrate. Poor bonding between the polymeric layer and the Mg implant may result in interfacial delamination under mechanical shearing (while inserting the implant) or long-term corrosive environment, which in turn compromises the protective properties of the coating [54]. The adhesion strength of SF and SF-CNC coatings to the AZ31 substrate and the effect of PD pretreatment on that was examined by ASTM D3359 cross-cut tape test. FIG. 7 depicts the photographs of cross-cut areas of SF and SF-CNC coated AZ31-OH and AZ31-PD substrates taken before and after the tape test. In the case of SF and SF-CNC coated AZ31-OH without PD, a relatively high percentage of the coatings were detached from the surface after tape removal, indicating an insufficient adhesion strength between the coatings and alkaline treated Mg alloy. In contrast, SF and SF-CNC coatings on PD-modified AZ31 showed superior adhesion, where the edges of the cuts were completely smooth and none of the squares were detached. Considering the number of detached squares (fully or partially), the adhesion strength of SF and SF-CNC coatings on AZ31-OH and AZ31-PD were graded as 2B and 5B, respectively. The improved adhesion of the SF and SF-CNC to the AZ31-PD can be attributed to the extraordinary robust adhesion of the PD to the Mg surface along with the existence of numerous functional groups (hydroxyl and amine) that can react with the coatings [60]. The application of PD as an intermediary layer to improve the adhesion of an external polymeric layer has been previously reported in several other studies [37, 61]. The results indicated that the SF and SF-CNC coatings have excellent adhesion strength on the PD pretreated AZ31 substrate, making them more feasible for clinical application.

### In Vitro Corrosion Studies

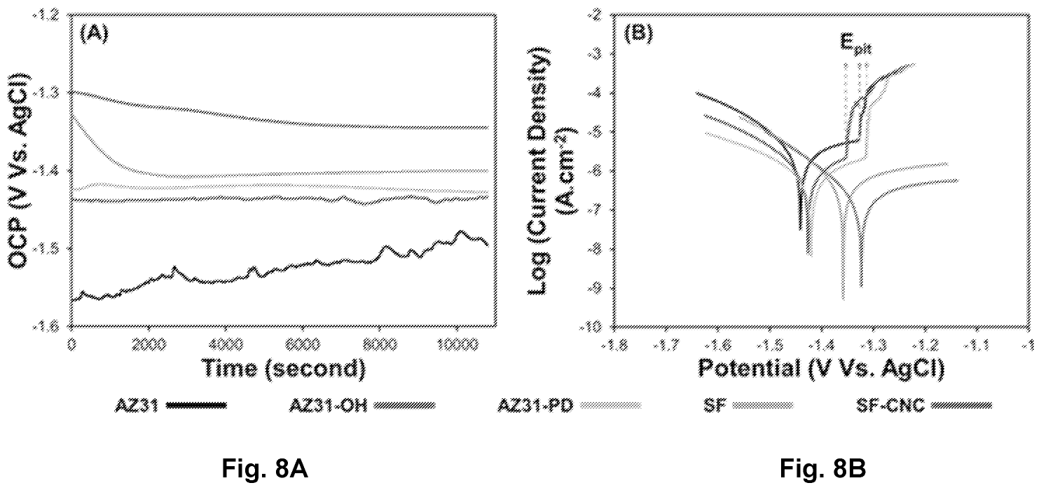

Electrochemical Measurements—Electrochemical corrosion tests including open circuit potential, potentiodynamic polarization, and electrochemical impedance spectroscopy were conducted in Hank's solution to evaluate the corrosion resistance properties of the coatings. FIG. 8A shows the OCP vs. time plots of the samples during 3 h of immersion. Generally, a more positive OCP value represents a nobler surface with less susceptibility to corrosion [62]. As expected, the bare AZ31 stabilized at a quite negative OCP of −1.49 V (vs. Ag/AgCl), which was the lowest value among all tested samples. For AZ31-OH, the steady OCP value was found to be −1.43 V, showing a more stable surface was achieved after alkaline treatment of Mg substrates. Deposition of PD film on the AZ31-OH did not noticeably change the final OCP value. The OCP of the SF-coated sample started from −1.33 V and finally reached −1.40 V at the end of test period. Although being much more positive than those uncoated Mg samples, the decreasing trend of OCP values during immersion time implies the deterioration of pure SF coating barrier properties, which ultimately may lead to the breakdown of the coating upon long exposure to Hank's solution [30]. Incorporation of CNC into the SF coating further increased the final OCP toward more positive value of −1.34 V, while mitigating the reduction trend of OCP during immersion time compared to that of pure SF coating. The OCP results suggested that CNC can act as an anodic protector, slowing down the cathodic hydrogen evolution and therefore, the overall anodic dissolution rate of the Mg alloy [63].

FIG. 8B displays the PP curves (Tafel plots) of uncoated and coated Mg samples, while the values of corrosion potential (E_{corr}) and corrosion current density (I_{corr}) derived from the Tafel plots are summarized in Table 2. Generally, curves with more positive E_{corr} and lower I_{corr} represent a sample with less corrosion tendency and corrosion rates [64]. The pristine AZ31 substrate had the lowest E_{corr} of −1.44 V and the highest I_{corr} of 2.8×10⁻⁶ A·cm⁻² among all the tested samples. Alkaline treatment of AZ31 resulted in a shift of E_{corr} toward a more positive potentials and reduction of I_{corr} to 8.9×10⁻⁷ A·cm⁻². This improvement in corrosion resistance is associated to the formed Mg(OH)₂ conversion layer [65]. Formation of PD on the AZ31-OH surface did not bring about any protective effect as no significant changes in E_{corr} and I_{corr} values were observed. It's worth mentioning that for all uncoated samples (AZ31, AZ31-OH, and AZ31-PD), sharp increases in the current density were observed in the anodic region of the polarization curves, implying these samples underwent pitting corrosion during the polarization test leading to sudden oxidation [66]. After deposition of SF coating, E_{corr} remarkably shifted to a more positive value of −1.35 V and I_{corr} was decreased to 5.7×10⁻⁷ A·cm⁻². This reflects the superior corrosion resistance of the SF coated sample, which is originated from the barrier properties of the compact silk film against the corrosive environment [44]. Incorporation of CNCs into the SF coating further shifted the E_{corr} value to −1.31 V and resulted in the lowest I_{corr} value of 2.5×10⁻⁷ A·cm⁻², which was approximately an order of magnitude lower than that of bare AZ31. The results of PP test clearly confirmed the superior effectiveness of the composite SF-CNC coating in retarding the rapid corrosion of the Mg substrate.

TABLE 2
Corrosion potentials (E_{corr}) and corrosion current densities
(i_{corr}) values of the Mg samples obtained from
PP curves in Hank's solution by Tafel methods.

| Samples | E_{corr} (V) | I_{corr} (A/cm²) |
|---|---|---|
| AZ31 | −1.44 | 2.8 × 10⁻⁶ |
| AZ31-OH | −1.42 | 8.9 × 10⁻⁷ |
| AZ31-PD | −1.41 | 8.7 × 10⁻⁷ |
| SF | −1.35 | 5.7 × 10⁻⁷ |
| SF-CNC | −1.31 | 2.5 × 10⁻⁷ |

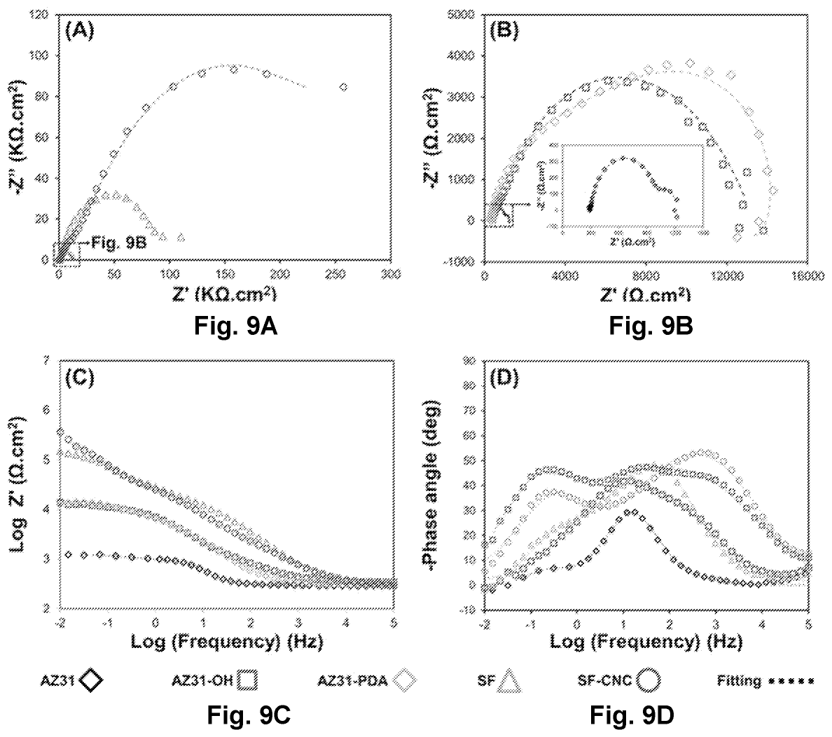

EIS was employed as a powerful, non-intrusive tool to further evaluate the corrosion protection properties of the coatings. FIGS. 9A-9C exhibit the Nyquist plots and Bode plots (both impedance and phase angle plots) of EIS spectra of the samples. In a typical Nyquist plot, a higher diameter of the impedance arc represents the superior corrosion resistance of a tested sample [9]. As can be seen in FIG. 9A-B, the arc diameters were found to be in the following order: AZ31<AZ31-OH ˜ AZ31-PD<SF<SF-CNC. Moreover, the impedance values at the lowest-frequency region (|Z|_{0.01 Hz}) of the Bode-impedance plots (FIG. 9C), which is also known to be a semi-quantitative indicative of anticorrosion performance, followed the same order. The results were consistent to those of observed in OCP and PP tests and clearly showed the protective properties of the SF coating on the Mg alloy, while confirming the enhancement effect of CNCs on the anticorrosion properties of the SF coating. As can be seen in the Bode-phase plots (FIG. 9D), the peak at the medium-frequency range became more eminent following the alkaline treatment of AZ31, indicating the enhancement of the oxide layer [67]. Moreover, coating the sample with the SF and SF-CNC resulted in loftier and wider phase angles, confirming the success of coating process [55]. Compared to the SF, SF-CNC exhibited broader time constants and kept higher impedance values over the low- and medium-frequency ranges (10⁻²-10² Hz), implying the superior corrosion protection performance of the SF-CNC coating [9].

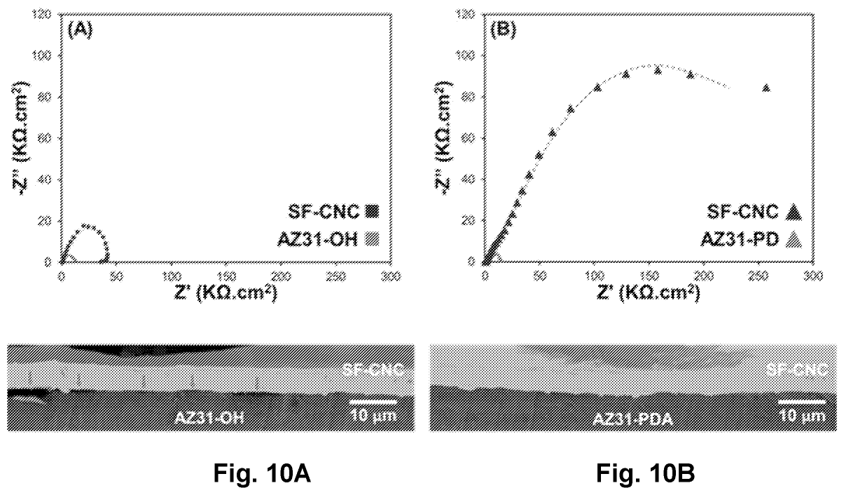

The effect of PD modification on the corrosion protection properties of the SF-CNC coating was also investigated through EIS measurement. FIGS. 10A-10B show the Nyquist EIS spectra of SF-CNC coating on the AZ31-OH surface with and without subsequent PD modification with the corresponding cross-sectional SEM images of the coating/substrate interfaces. As can be seen, the diameter of semicircle only slightly increased following the application of SF-CNC coating on AZ31-OH, implying a very limited improvement in corrosion protection offered by the coating. Moreover, an inductive loop appeared on the Nyquist spectrum at the low-frequency region, which is attributed to the pitting corrosion and adsorption/desorption of intermediates on the metal substrate [68]. The poor corrosion resistance of the SF-CNC coating on AZ31-OH is most likely because of the weak bonding between the polymer layer and the underneath metal substrate, which could result in interfacial delamination and loss of corrosion protection properties [69]. This assumption is further supported by the high magnified cross-sectional SEM images of the SF-CNC coating, where several cleavages were observed sporadically along the metal/coating interface, when the SF-CNC coating was applied on the AZ31-OH surface with no subsequent PD modification. In contrast, in the case of SF-CNC coating on the PD-modified AZ31-OH, the diameter of impedance arc was remarkably increased. Besides, no inductive loop was observed at the lower frequencies, suggesting the presence of a stable corrosion protective layer on the metal surface [70]. A similar enhancement effect of intermediary PD layer on the corrosion protection performance of an externally applied polymeric coating has been previously reported in several studies and attributed to the higher adhesion at substrate/coating interface resulted from the chemical interactions as well as mechanical anchoring between the organic coating and the PD surface [61, 69, 71, 72]. The results clearly indicated that the presence of the intermediary PD layer with improved adhesiveness to the secondary coating is decisive to acquire a notable corrosion protection offered by the SF-CNC coating.

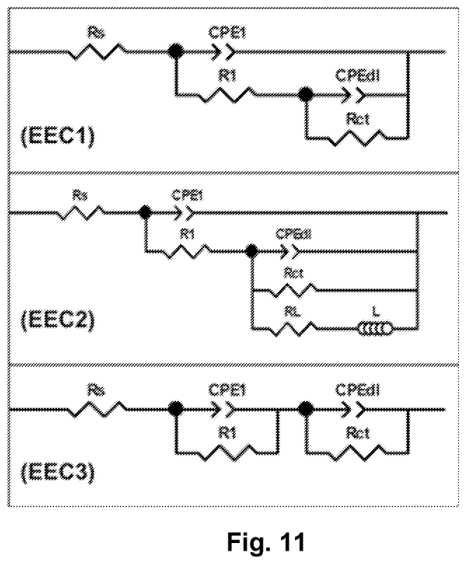

Appropriate electrical equivalent circuits (EECs) (FIG. 11) were employed for each group of the samples to further analyze the EIS results, and the electrochemical parameters from using equivalent circuit fitting of each model are summarized in Table 3. The EEC1 used for bare AZ31 and AZ31-OH was expressed as Rₛ(CPE₁(CPE_{di}R_{ct})R₁) to describe a surface with an outer oxide layer due to natural oxidation of the Mg alloy or alkaline treatment [73], where Rₛ represents the electrolyte resistance, R₁ and CPE₁ represent the resistance and capacitance of the outer layer, and R_{ct} and CPE_{di} appear for the interfacial charge transfer resistance and electric double-layer capacitance, respectively. In the case of AZ31-PD, an R_{L}-L component was implemented and the EEC2 was expressed as Rₛ(CPE₁(CPE_{di} R_{ct} R_{L}-L)R₁) to account for the pseudo-inductive behavior at the low-frequency region, which is reported to be associated with the adsorption/desorption of intermediates on the metal substrate dissolution and pitting corrosion [74]. For the SF and SF-CNC coated samples, the employed EEC3 consists of two equivalent circuits in series expressed as Rₛ(CPE₁ R₁)(CPE_{di} R_{ct}), where CPE₁ and R₁ represent capacitive behavior and the resistance of the barrier coatings, respectively. It should be mentioned that constant-phase elements were implemented in all EEC models instead of ideal capacitors to reflect the intrinsic inhomogeneity of the coatings and the metal surface as well as minimizing the fitting error [9]. According to Table 3, the highest R_{ct} value (31.2 kΩ·cm²) was found for SF-CNC, implying the highest impedance of the corrosion reaction and therefore the best corrosion protection performance. Moreover, SF-CNC had also the highest R₁ among all the samples, indicating the superior barrier properties of the composite SF-CNC coating.

TABLE 3
Representative fitting results of EIS spectra of Mg samples using appropriate equivalent electrical circuits.

| | Rₛ | R₁ | Q₁ | | R_{ct} | Q_{dl} | | R_{L} | L | |
|---|---|---|---|---|---|---|---|---|---|---|
| Samples | (Ω · cm²) | (Ω · cm²) | (F · cm⁻²) | n₁ | (Ω · cm²) | (F · cm⁻²) | n₂ | (Ω · cm²) | (H · cm²) | Chi² |
| AZ31 | 2.91 × 10² | 7.56 × 10² | 4.26 × 10⁻⁵ | 0.89 | 1.71 × 10² | 4.36 × 10⁻³ | 0.99 | — | — | 0.0050 |
| AZ31- | 3.28 × 10² | 6.75 × 10³ | 2.37 × 10⁻⁵ | 0.61 | 6.22 × 10³ | 1.19 × 10⁻⁶ | 0.99 | — | — | 0.0027 |
| OH | | | | | | | | | | |
| AZ31- | 3.02 × 10² | 6.84 × 10³ | 1.14 × 10⁻⁵ | 0.75 | 8.27 × 10³ | 8.63 × 10⁻⁵ | 0.75 | 3.04 × 10⁴ | 2.31 × 10⁵ | 0.0021 |
| PD | | | | | | | | | | |
| SF | 2.69 × 10² | 1.41 × 10⁵ | 1.19 × 10⁻⁵ | 0.71 | 1.56 × 10⁴ | 2.19 × 10⁻⁶ | 0.70 | — | — | 0.0021 |
| SF- | 2.54 × 10² | 2.63 × 10⁶ | 1.33 × 10⁻⁵ | 0.78 | 3.12 × 10⁴ | 8.52 × 10⁻⁶ | 0.57 | — | — | 0.0014 |
| CNC | | | | | | | | | | |

Immersion Corrosion Tests—Immersion tests were carried out to further study the long-term degradation behavior of the bare and modified Mg alloys over a period of 14 days. Since pH increment of the corrosion media and evolution of hydrogen gas co-occur with the corrosion of Mg alloys (according to following equation), they can be used as reliable corrosion indicators of Mg-based alloys [75].

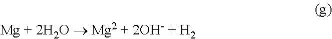

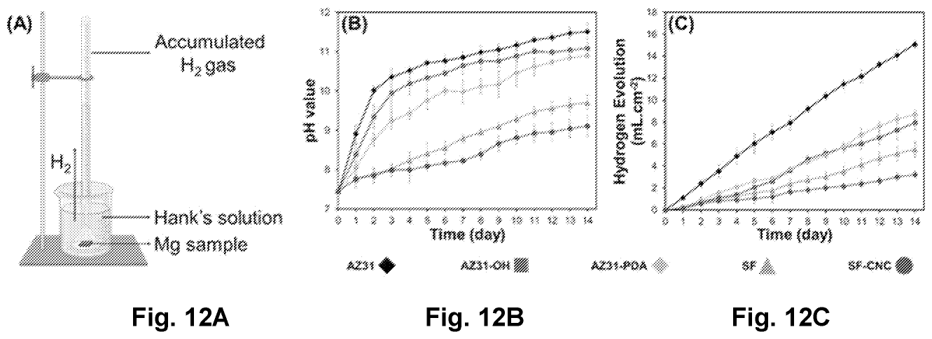

As shown in FIG. 12B, in the case of bare AZ31, the pH value of the corrosion media at the end of the 14-day immersion test was found to be 11.50±0.20, which was significantly higher than 11.08±0.14 for the AZ31-OH, 10.90±0.07 for the AZ-PD, 9.71±0.19 for the SF, and 9.11±0.26 for the SF-CNC samples, showing a very high reactivity and rapid corrosion of the unmodified AZ31 alloy. A similar trend was observed in the hydrogen evolution measurements (FIG. 12C). The AZ31 substrate released the highest volume of hydrogen (15.08±0.33 mL·cm⁻²) among all samples after 14 days of immersion, while the final accumulated H₂ volumes were found to be 7.90±0.68 mL·cm⁻², 8.70±0.40 mL·cm⁻², 5.51±0.70 mL·cm⁻², and 3.22±0.31 mL·cm⁻² for AZ31-OH, AZ31-PD, SF, and SF-CNC, respectively. The results clearly indicated the superior corrosion protection offered by SF-based coatings, which became more significant after incorporation of CNC into the coatings. Like the SF-CNC coating, the pure SF coating was quite effective in slowing down the alkalization and hydrogen evolution, but only during the initial days. By increasing the immersion time, the pH values and the hydrogen generation rates started to increase and deviated from those of SF-CNC samples. Although being able to render a temporary protection as a result of a thick and dense polymer layer, it is evident that the pure SF coating cannot provide a steady and prolonged protection against corrosion.

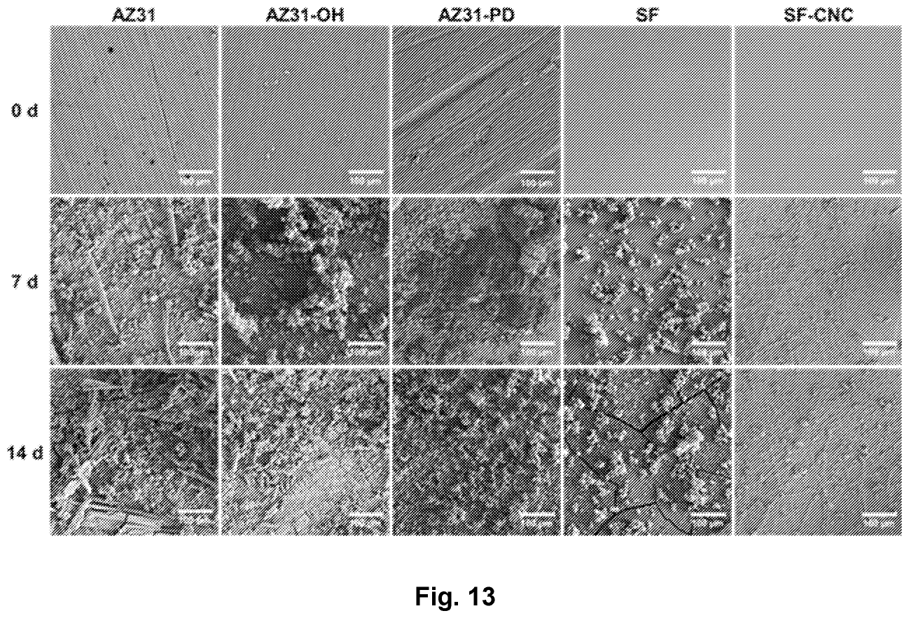

SEM was employed to observe the post-corrosion surface morphology and comparatively evaluate the severity of corrosion on different samples during 14 days of immersion in Hank's solution (FIG. 13). The post-corrosion SEM images were found to be in a good agreement with the results of pH and hydrogen evolution measurements. As can be seen, thick layers of corrosion products cumulatively formed on the surfaces of uncoated AZ31, AZ31-OH, AZ31-PD samples after 7 and 14 days of immersion, implying that they severely corroded during the immersion test. For the SF coating, although much smaller amount of corrosion products was found on the surface compared to those of uncoated samples on day 7, there still appeared several cracks on the surface. Emergence of these wide cracks is extremely detrimental for the protective coating, as it weakens the barrier properties in long-term and results in the exposure of the underlying Mg substrate to the corrosive electrolyte [76]. Therefore, severe signs of corrosion were observed on the SF surface after 14 days, like those occurred on the uncoated samples surfaces. In contrast, the SF-CNC presented a more uniform and intact surface with much less apparent corrosion products after 14 days of immersion. Although there were still some tiny cracks appeared on the coating, they were much slender than those appeared on the pure SF coating. This is most likely due to the higher content of 3-sheet in the SF-CNC along with the inherent anticorrosion properties of the CNC, which will be discussed in detail in section 3.5. Overall, the results of electrochemical corrosion measurements and in vitro immersion study demonstrated that the SF-CNC coating has superior barrier properties and can effectively enhance the corrosion resistance and provide a prolonged protection for the underlying AZ31 substrate against corrosion.

Mechanism of corrosion resistance enhancement of CNC—Different mechanisms have been proposed to explain the improved corrosion resistance of CNC-containing composite coatings. He et al. has found that the incorporation of CNC into a waterborne hydroxyacrylate-based coating decreased the water uptake capacity of the coating due to the strong hydrogen bonding between CNC and polymeric matrix, and thus retards the penetration of water and corrosive species to the metal surface [27]. Similarly, the results of ATR-FTIR in this study also suggested the presence of strong hydrogen bonding between the SF matrix and CNCs. It was also proposed by the same group that the formation of complexes between the released metal cations and the anionic half ester sulfate groups on CNCs may also contribute in enhancing corrosion resistance [29]. Another possible mechanism behind the anticorrosion enhancement effects of CNCs may lay on the concept of indirect paths. It has been hypothesized that homogeneously dispersed fillers, in our case CNCs, can fill up the porosity and possible structural defects of the polymeric matrices and force the corrosive species to traverse a lengthier and more indirect pathway through the composite coating to reach the metal surface, thus enhancing corrosion resistance of the coating [30, 77]. In addition to the all above mentioned mechanisms, the higher crystallinity (β-sheets percentage) of the SF-CNC coating (based on the results of deconvolution analysis) is likely another possible reason behind the improved corrosion resistance properties of this coating compared to the blank SF coating, as the superior water insolubility and corrosion resistance of the SF-based coatings with higher crystallinity percentages have been previously reported in other studies [14, 41, 78].

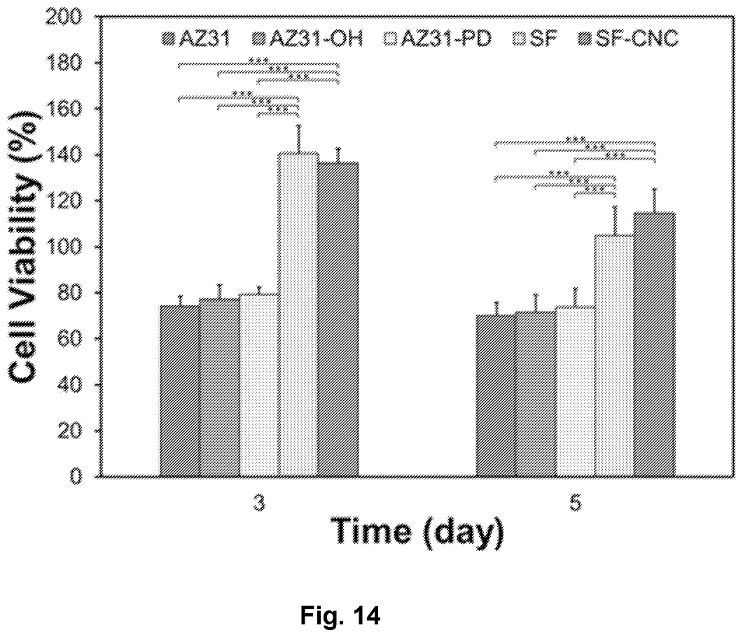

In vitro Cytocompatibility—Biocompatibility is a primary requirement for any material intended to be used as bio-implant. In vitro cytocompatibility was investigated as an initial criterion in the assessment of biocompatibility [79] by measuring the viability of hFOB cells exposed to leachates of the samples and observing their morphology after adhesion to the samples. FIG. 14 presents the viability percentage of hFOBs cultured with the 3-day and 5-day leachates from different samples. For uncoated Mg samples (AZ31, AZ31-OH, and AZ31-PD), the viabilities were relatively low (<80%) at both intervals, showing that the surfaces were unfavorable for hFOB cell growth. Moreover, no significant differences in viability percentages of uncoated samples were observed at any time intervals, indicating that none of these surface modifications (alkaline treatment and PD deposition) can remarkably enhance the cytocompatibility of the AZ31 alloy. In contrast, the viabilities of SF and SF-CNC coated samples were significantly higher than those of uncoated samples at both 3-day and 5-day intervals (P-value <0.001). Having high viabilities (above 100%) at both time points indicated that not only the SF and SF-CNC coatings were not cytotoxic but also, they could support the growth and proliferation of hFOB cells. Besides, no distinct difference between the viability of SF and SF-CNC was observed at any incubation times, suggesting the non-toxic nature of CNC as a bio-safe anticorrosion filler for the SF coating.

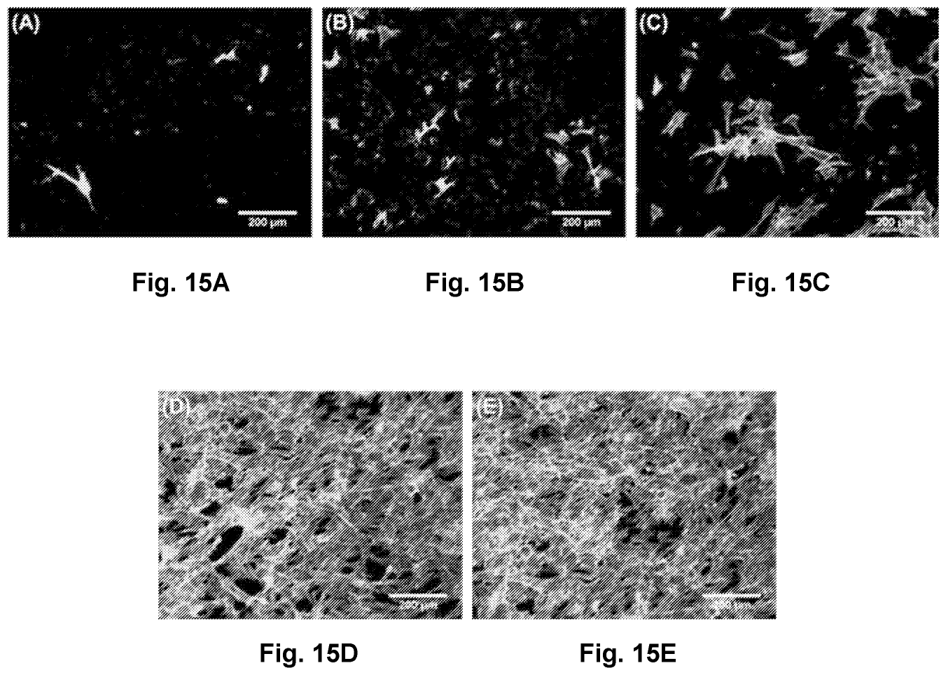

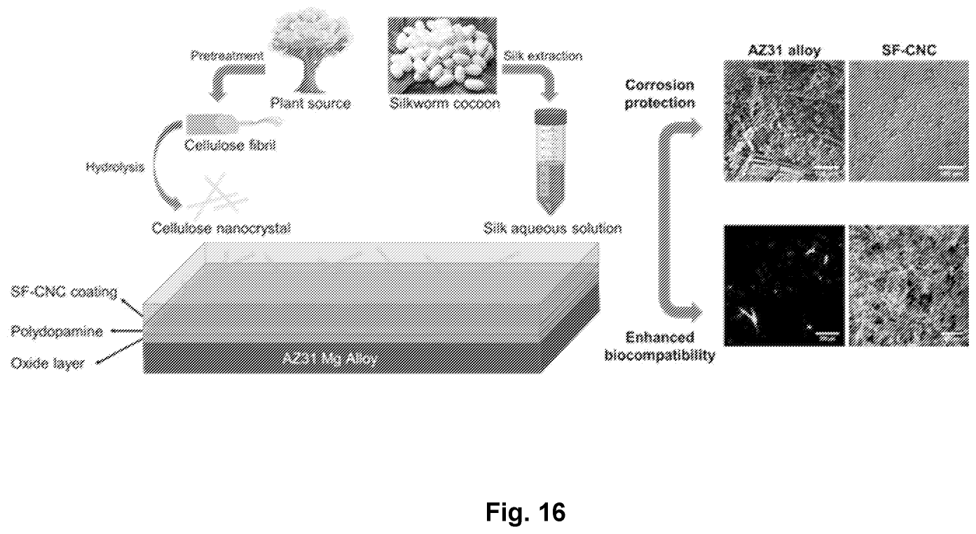

FIGS. 15A-15E show the fluorescence staining images of hFOB cells cultured on different substrates for 24 h. As can be seen, the number of cells adhered to SF and SF-CNC coatings is much higher compared to those of uncoated samples, almost covering all over the surfaces. Moreover, obvious differences in cellular morphologies were observed on the various surfaces. While the cells adhered and elongated well on the SF and SF-CNC surfaces with extended morphologies, the cells on the uncoated samples exhibited a much less striated or, in some cases, a round-shape morphology. This observation suggested that SF and SF-CNC coatings enhanced cellular adhesion and spreading on the Mg substrates. The improved viability and cellular adhesion of the SF and SF-CNC coatings most likely arises from the hampered rapid degradation of the Mg alloy along with the excellent biocompatibility of the SF itself. The presence of protective coatings prevents the local alkalization, increased concentration of Mg²⁺, and formation of excessive hydrogen gas within the culture media, which may otherwise damage transport channels of the cell membranes and cause cell necrosis [80]. Moreover, the favorable biocompatibility of SF toward bone cells as being reported previously in several studies [10, 41, 81, 82], likely contributes to the enhanced cellular responses observed in the coated samples. A schematic representation of the coating structure is shown in FIG. 16, along with representative SEM and fluorescence images of the SF-CNC coating compared to an uncoated AZ31 alloy. Altogether, the results clearly indicated that coating of AZ31 alloy with the SF and SF-CNC coatings remarkably enhanced the cytocompatibility of the bare Mg substrates both in terms of proliferation and adhesion.

### CONCLUSIONS

The multifunctional nanocomposite coating described herein incorporate CNCs into SF. The composite coating significantly improved the barrier and corrosion protection properties. The property enhancement could be attributed to the higher 3-sheets content of CNC-reinforced SF coating as well as the intrinsic properties of CNC as an anti-corrosive nanofiller. Surface pretreatment of AZ31 substrate with polydopamine before applying the protective coating was found to be necessary to achieve a notable corrosion protection by the coating. In addition to the corrosion resistance properties, applying SF-CNC coating significantly enhanced the biocompatibility of AZ31 alloy with regards to cell viability and adhesion/spreading toward human fetal osteoblast cells. Taken all together, the natural CNC-reinforced SF coating with excellent biological and enhanced anticorrosion properties holds a great potential as an ideal candidate for surface modification of Mg alloys in bio-implant applications.

### Additional Characterization Data for Silk Fibroin-Cellulose Nanocrystal (SF-CNC) Coating on AZ31 Mg Alloy

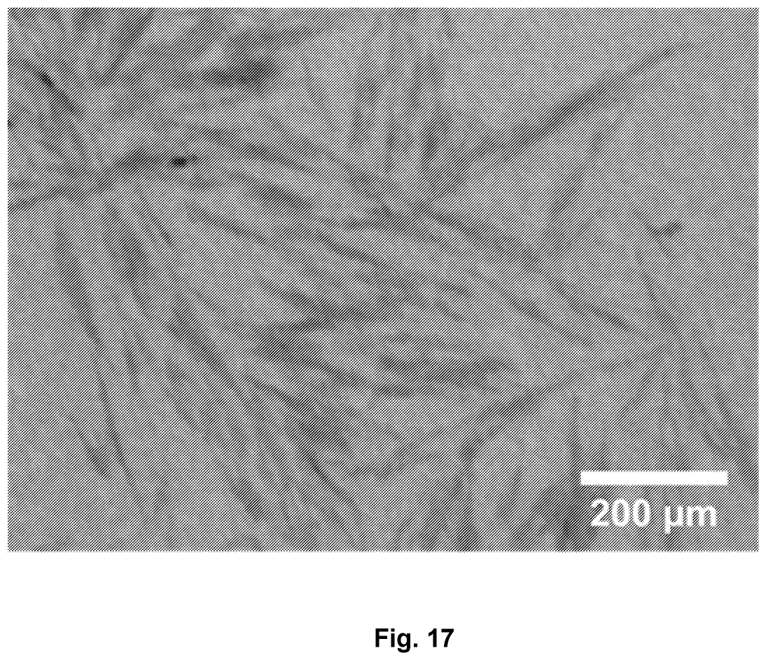

TEM imaging was performed for morphological and size analyses of CNCs using a JEOL JEM-2100 transmission electron microscope. FIG. 17 shows the typical rod-like structure of the disclosed CNCs having a cross-sectional dimension of about 21±5 nm and length of about 147±45 nm.

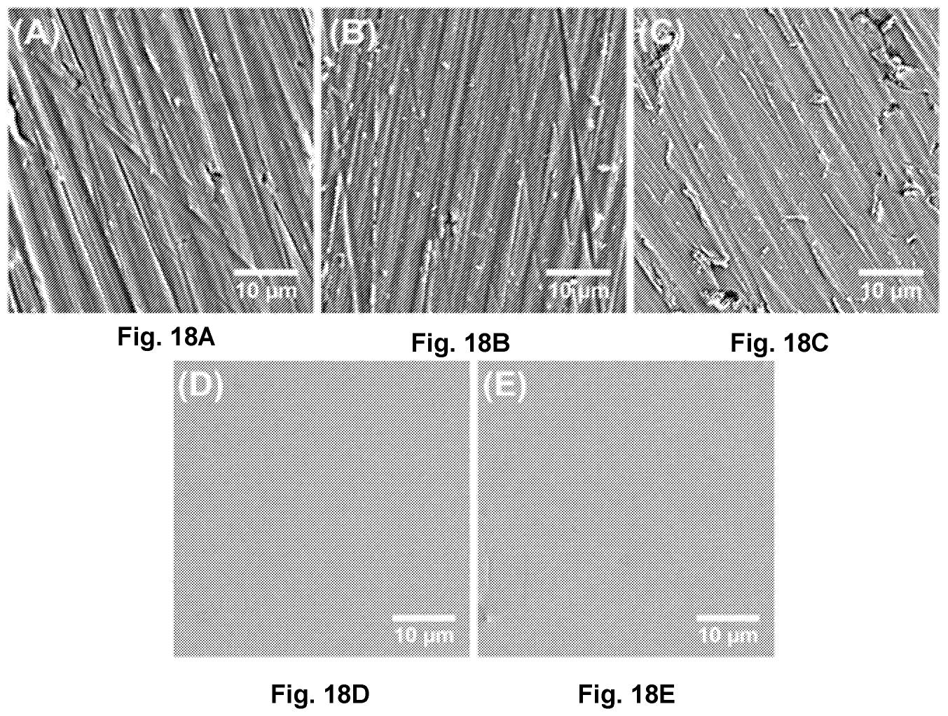

Surface SEM was also performed on as-prepared AZ31 (FIG. 18A), AZ31-OH (FIG. 18B), AZ31-PD (FIG. 18C), SF (FIG. 18D), and SF-CNC (FIG. 18E). SEM images with higher magnification (2000×) were taken from all the samples including coated ones (as prepared and before immersion test). FIGS. 18D-18E show that both SF and SF-CNC coatings have flat, smooth, and uniform surfaces with no apparent defects on them.

Long-term EIS Study

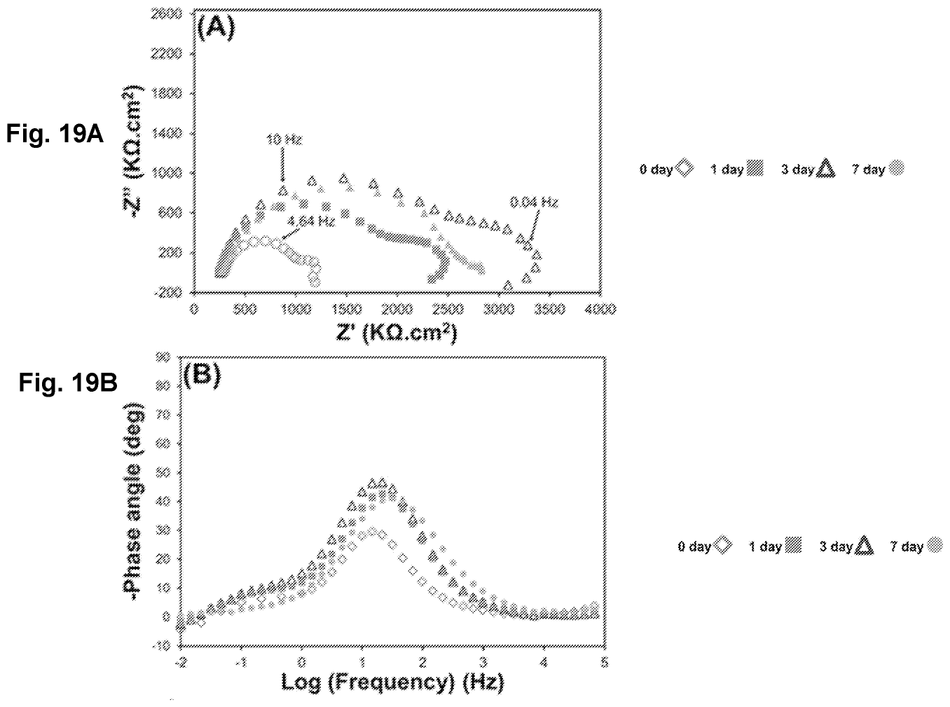

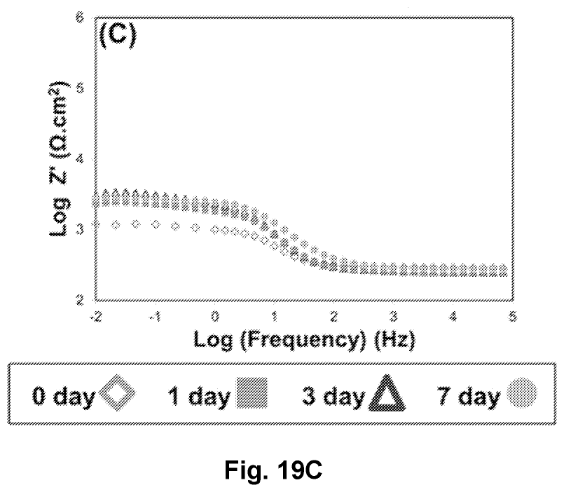

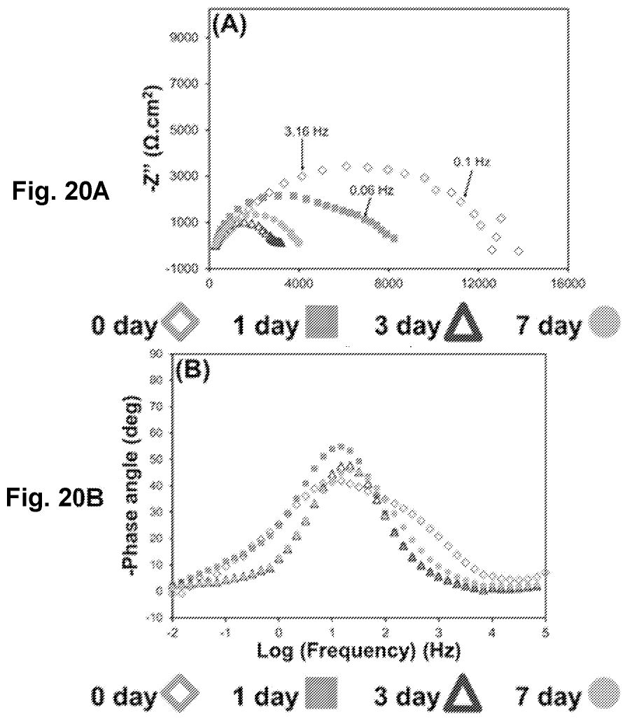

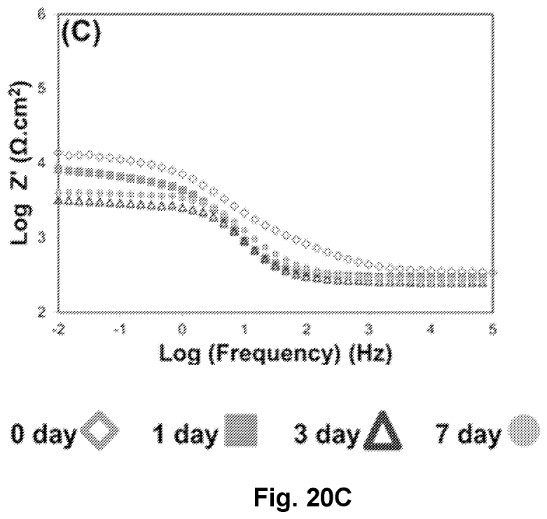

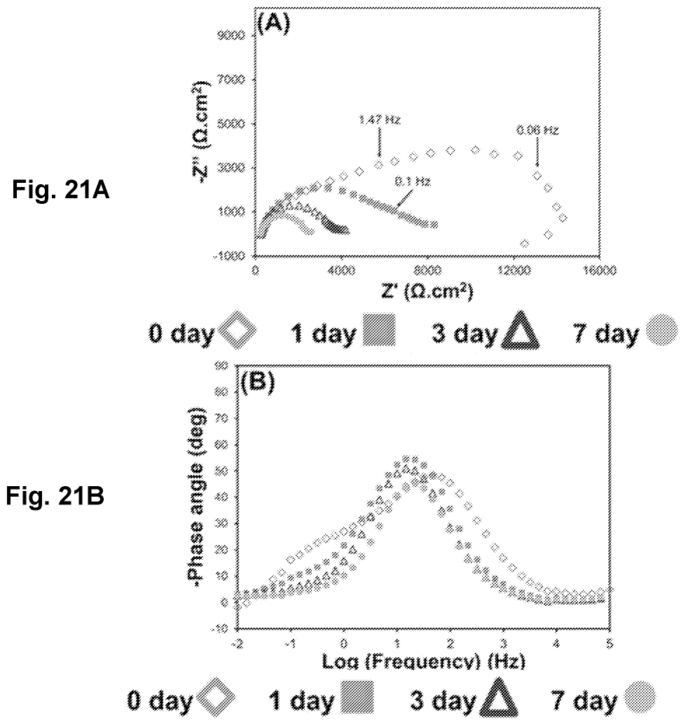

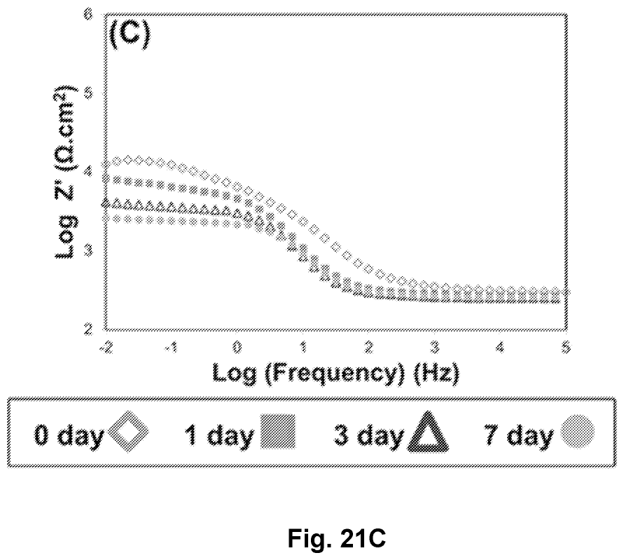

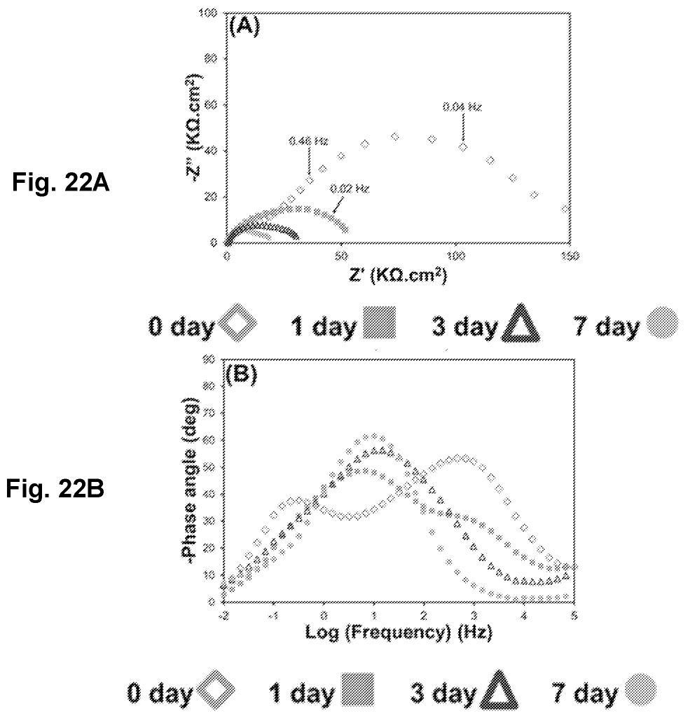

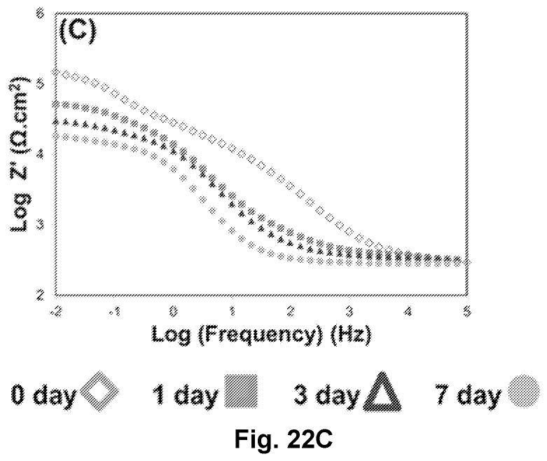

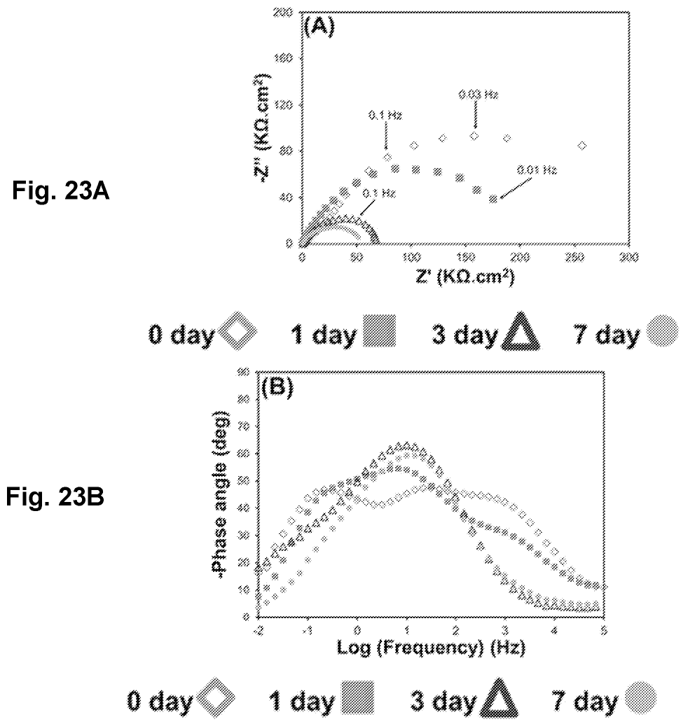

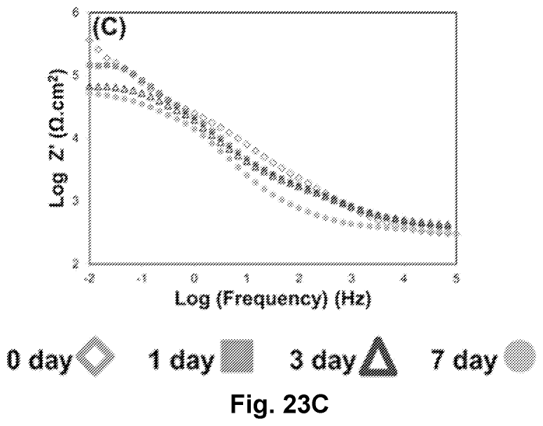

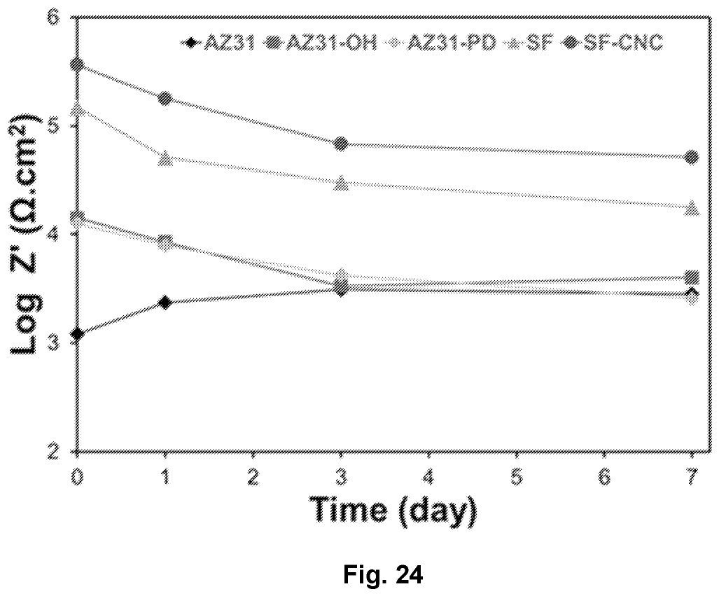

To further understand the long-term corrosion behavior, the samples were studied by EIS as a non-destructive technique during a 7-day immersion test in Hank's solution. Nyquist and Bode plots of the samples after 0, 1, 3, and 7 days of immersion are presented in FIGS. 19A-23C. |Z|_{0.01 Hz} values were employed to investigate the overall corrosion resistance of the sample at different time intervals, while a higher |Z|_{0.01 Hz} value generally reflects a lower corrosion rate of the corroding metal [83]. As shown in FIG. 24, the |Z|_{0.01 Hz} of bare AZ31 surface slightly increased by time and kept the same level of resistance until the end of the test, which can be attributed to the formation of surface corrosion products (mainly phosphates [9]) that can serve as a barrier against the corrosion medium. However, this layer is not strong enough to resist the aggressive corrosion process because of its loose and porous structure [84]. For AZ31-OH, the |Z|_{0.01 Hz} values continuously dropped within the immersion time. Although the formed Mg(OH)₂ layer from the alkaline treatment can initially improve the corrosion resistance by isolating the Mg substrate from the corrosion medium, with the presence of chloride ions in the corrosion electrolyte, the oxide layer will be converted into a much less stable MgCl₂ layer with poor barrier properties, resulting in a constant drop of impedance value overtime [85]. AZ31-PD showed also a similar decreasing trend in corrosion resistance like AZ31-OH, indicating that the deposited PD layer is not able to protect the metal surface against the corrosive ions and species present in the media. In the case of coated samples (SF and SF-CNC), the |Z|_{0.01 Hz} values started to decline with prolonged immersion time most likely due to the gradual diffusion of water and corrosive species into the coatings and decrement of the physical barrier properties over time. Similar observations have been reported in previous studies for Mg alloys coated with SF-based coatings [10, 55, 8, 81]. Compared to the SF, the SF-CNC showed less variations in |Z|_{0.01 Hz} values during the immersion period while it had the highest |Z|_{0.01 Hz} among all the samples at the end of the immersion test, showing its superior long-term barrier properties and durable corrosion protection. The degradation mechanism of the coated samples can be explained as follows. Following the immersion of coated samples in Hank's solution, water and corrosive species start penetrating through the coatings and cause volume expansion and subsequently swelling of the coating [8]. Meanwhile, the generation of H₂ gas resulting from the reaction of Mg and water molecules on the metal surface further contributes and results in the emergence of narrow cracks on the SF coating [55]. With the increasing of immersion time, the cracks become broader, resulting in increased metal surface exposure to the media and further corrosion. Nonetheless, the composite SF-CNC coating, owing to the enhanced barrier properties as a results of CNC incorporation can mitigate the penetration of water and corrosive ions through the coating, hence lower the corrosion reaction rate of the underneath Mg substrate. The results of the in vitro immersion corrosion tests including the post-corrosion surface SEM images of the coated samples, pH change, and hydrogen evolution measurements were all found to be in agreement with the proposed mechanism.

### Aspects of the Disclosure

The present disclosure will be better understood upon reading the following numbered aspects, which should not be confused with the claims. Any of the numbered aspects below can, in some instances, be combined with aspects described elsewhere in this disclosure and such combinations are intended to form part of the disclosure.

Aspect 1. A biodegradable orthopedic implant, comprising an implant comprising a Mg alloy and a coating comprising silk fibroin.

Aspect 2. The biodegradable orthopedic implant of aspect 1, wherein the coating comprises a first layer, and a second layer; wherein the first layer comprises polydopamine; and wherein the second layer comprises silk fibroin.

Aspect 3. The biodegradable orthopedic implant of aspect 2, wherein the second layer further comprises a biocompatible polymer.

Aspect 4. The biodegradable orthopedic implant of aspect 3, wherein the biocompatible polymer is cellulose nanocrystal.

Aspect 5. The biodegradable orthopedic implant of aspects 3 or 4, wherein the biocompatible polymer and silk fibroin form a composite.

Aspect 6. The biodegradable orthopedic implant of any of the preceding aspects, wherein an oxide layer is disposed on an external surface of the implant.

Aspect 7. The biodegradable orthopedic implant of any of the preceding aspects, wherein the implant comprises a Mg—Al—Zn alloy.

Aspect 8. The biodegradable orthopedic implant of aspect 7, wherein the Mg—Al—Zn alloy has an Al content of about 3%.

Aspect 9. The biodegradable orthopedic implant of aspects 7 or 8, wherein the Mg—Al—Zn alloy is AZ31.

Aspect 10. The biodegradable orthopedic implant of aspect 4, wherein the second layer comprises about 0.5-3.0% (w/w) cellulose nanocrystal with respect to silk fibroin weight.

Aspect 11. The biodegradable orthopedic implant of any of aspects 3-10, wherein the first layer has sub-micrometer thickness, and wherein the second layer is from about, 8 μm to 14 μm thick or about 8.7 μm to 13.7 μm thick.

Aspect 12. The biodegradable orthopedic implant of any of the preceding aspects, wherein oxide layer is from about 2 μm to 20 μm thick.

Aspect 13. A corrosion-resistant coating for orthopedic implants, comprising a first layer comprising polydopamine and a second layer comprising silk fibroin.

Aspect 14. The corrosion-resistant coating of aspect 13, wherein the second layer further comprises cellulose nanocrystals.

Aspect 15. The corrosion-resistant coating of aspects 13 or 14, wherein the coating is disposed on an orthopedic implant, the orthopedic implant having an oxide layer on an external surface.

Aspect 16. The corrosion-resistant coating of aspect 13, wherein the coating is disposed on an external surface of an orthopedic implant.

Aspect 17. A method of making a coated orthopedic implant, comprising immersing a magnesium alloy implant in an alkaline solution to form an oxide-coated implant; providing a layer comprising polydopamine on the oxide-coated implant; and providing a coating comprising silk fibroin over the layer comprising polydopamine.

Aspect 18. The method of aspect 17, wherein the coating comprising silk fibroin further comprises cellulose nanocrystals.

Aspect 19. The method of aspects 17 or 18, further comprising applying a second coating comprising silk fibroin to the implant.

Aspect 20. The method of any of aspects 17-19, wherein the alkaline solution is about 0.1-5 M NaOH.

Aspect 21. The method of any of aspects 18-20, wherein the wherein the silk fibroin layer comprises about 0.5-3.0% (w/w) cellulose nanocrystal with respect to silk fibroin weight.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

### REFERENCES

[1] J. L. Wang, J. K. Xu, C. Hopkins, D. H. K. Chow, L. Qin, Biodegradable magnesium-based implants in orthopedics—a general review and perspectives, Advanced science, 7 (2020) 1902443.
[2] K.-H. Cheon, C. Park, M.-H. Kang, I.-G. Kang, M.-K. Lee, H. Lee, H.-E. Kim, H.-D. Jung, T.-S. Jang, Construction of tantalum/poly (ether imide) coatings on magnesium implants with both corrosion protection and osseointegration properties, Bioactive materials, 6 (2021) 1189-1200.
[3] V. Jothi, A. Y. Adesina, A. M. Kumar, M. M. Rahman, J. N. Ram, Enhancing the biodegradability and surface protective performance of AZ31 Mg alloy using polypyrrole/gelatin composite coatings with anodized Mg surface, Surface and Coatings Technology, 381 (2020) 125139.
[4] M. Toorani, M. Aliofkhazraei, M. Mahdavian, R. Naderi, Effective PEO/Silane pretreatment of epoxy coating applied on AZ31B Mg alloy for corrosion protection, Corrosion Science, 169 (2020) 108608.
[5] L.-Y. Li, L.-Y. Cui, R.-C. Zeng, S.-Q. Li, X.-B. Chen, Y. Zheng, M. B. Kannan, Advances in functionalized polymer coatings on biodegradable magnesium alloys-a review, Acta biomaterialia, 79 (2018) 23-36.
[6] S. Kunjukunju, A. Roy, M. Ramanathan, B. Lee, J. E. Candiello, P. N. Kumta, A layer-by-layer approach to natural polymer-derived bioactive coatings on magnesium alloys, Acta biomaterialia, 9 (2013) 8690-8703.
[7] S. Heise, S. Virtanen, A. R. Boccaccini, Tackling Mg alloy corrosion by natural polymer coatings-A review, Journal of Biomedical Materials Research Part A, 104 (2016) 2628-2641.
[8] P. Xiong, Z. Jia, M. Li, W. Zhou, J. Yan, Y. Wu, Y. Cheng, Y. Zheng, Biomimetic Ca, Sr/P-doped silk fibroin films on Mg-1Ca alloy with dramatic corrosion resistance and osteogenic activities, ACS Biomaterials Science & Engineering, 4 (2018) 3163-3176.
[9] H. Asadi, B. Suganthan, S. Ghalei, H. Handa, R. P. Ramasamy, A multifunctional polymeric coating incorporating lawsone with corrosion resistance and antibacterial activity for biomedical Mg alloys, Progress in Organic Coatings, 153 (2021) 106157.
[10] P. Xiong, J. Yan, P. Wang, Z. Jia, W. Zhou, W. Yuan, Y. Li, Y. Liu, Y. Cheng, D. Chen, A pH-sensitive self-healing coating for biodegradable magnesium implants, Acta biomaterialia, 98 (2019) 160-173.
[11] C. Wang, Z. Yi, Y. Sheng, L. Tian, L. Qin, T. Ngai, W. Lin, Development of a novel biodegradable and anti-bacterial polyurethane coating for biomedical magnesium rods, Materials Science and Engineering: C, 99 (2019) 344-356.
[12] W. Xu, K. Yagoshi, Y. Koga, M. Sasaki, T. Niidome, Optimized polymer coating for magnesium alloy-based bioresorbable scaffolds for long-lasting drug release and corrosion resistance, Colloids and Surfaces B: Biointerfaces, 163 (2018) 100-106.
[13] P. Makkar, H. J. Kang, A. R. Padalhin, I. Park, B.-G. Moon, B. T. Lee, Development and properties of duplex MgF2/PCL coatings on biodegradable magnesium alloy for biomedical applications, Plos one, 13 (2018) e0193927.
[14] C. Wang, H. Fang, X. Qi, C. Hang, Y. Sun, Z. Peng, W. Wei, Y. Wang, Silk fibroin film-coated MgZnCa alloy with enhanced in vitro and in vivo performance prepared using surface activation, Acta biomaterialia, 91 (2019) 99-111.
[15] S.-H. Ye, Y. Chen, Z. Mao, X. Gu, V. Shankarraman, Y. Hong, V. Shanov, W. R. Wagner, Biodegradable zwitterionic polymer coatings for magnesium alloy stents, Langmuir, 35 (2018) 1421-1429.
[16] Y. Li, S. Zhao, S. Li, Y. Ge, R. Wang, L. Zheng, J. Xu, M. Sun, Q. Jiang, Y. Zhang, Surface engineering of biodegradable magnesium alloys for enhanced orthopedic implants, Small, 15 (2019) 1904486.
[17] P. Rahmani, A. Shojaei, N. P. Tavandashti, Nanodiamond loaded with corrosion inhibitor as efficient nanocarrier to improve anticorrosion behavior of epoxy coating, Journal of Industrial and Engineering Chemistry, 83 (2020) 153-163.
[18] R. S. EI-Kamel, A. A. Ghoneim, A. M. Fekry, Electrochemical, biodegradation and cytotoxicity of graphene oxide nanoparticles/polythreonine as a novel nano-coating on AZ91E Mg alloy staple in gastrectomy surgery, Materials Science and Engineering: C, 103 (2019) 109780.
[19] A. Zomorodian, M. Garcia, T. M. e Silva, J. Fernandes, M. H. Fernandes, M. d. F. Montemor, Biofunctional composite coating architectures based on polycaprolactone and nanohydroxyapatite for controlled corrosion activity and enhanced biocompatibility of magnesium AZ31 alloy, Materials Science and Engineering: C, 48 (2015) 434-443.
[20] Y. Guo, S. Jia, L. Qiao, Y. Su, R. Gu, G. Li, J. Lian, A multifunctional polypyrrole/zinc oxide composite coating on biodegradable magnesium alloys for orthopedic implants, Colloids and Surfaces B: Biointerfaces, 194 (2020) 111186.
[21] M.-H. Kang, T.-S. Jang, H.-D. Jung, S.-M. Kim, H.-E. Kim, Y.-H. Koh, J. Song, Poly (ether imide)-silica hybrid coatings for tunable corrosion behavior and improved biocompatibility of magnesium implants, Biomedical Materials, 11 (2016) 035003.
[22] A. A. Nazeer, E. AI-Hetlani, M. O. Amin, T. QuiAones-Ruiz, I. K. Lednev, A poly (butyl methacrylate)/graphene oxide/TiO2 nanocomposite coating with superior corrosion protection for AZ31 alloy in chloride solution, Chemical Engineering Journal, 361 (2019) 485-498.
[23] Y.-j. Shi, J. Pei, J. Zhang, J.-I. Niu, H. Zhang, S.-r. Guo, Z.-h. Li, G.-y. Yuan, Enhanced corrosion resistance and cytocompatibility of biodegradable Mg alloys by introduction of Mg (OH) 2 particles into poly (L-lactic acid) coating, Scientific reports, 7 (2017) 1-11.
[24] H. Li, F. Peng, D. Wang, Y. Qiao, D. Xu, X. Liu, Layered double hydroxide/poly-dopamine composite coating with surface heparinization on Mg alloys: improved anticorrosion, endothelialization and hemocompatibility, Biomaterials science, 6 (2018) 1846-1858.
[25] J. Huang, L. Liu, J. Yao, Electrospinning of Bombyx mori silk fibroin nanofiber mats reinforced by cellulose nanowhiskers, Fibers and Polymers, 12 (2011) 1002-1006.
[26] C. Calvino, N. Macke, R. Kato, S. J. Rowan, Development, processing and applications of bio-sourced cellulose nanocrystal composites, Progress in Polymer Science, 103 (2020) 101221.
[27] Y. He, Y. Boluk, J. Pan, A. Ahniyaz, T. Deltin, P. M. Claesson, Corrosion protective properties of cellulose nanocrystals reinforced waterborne acrylate-based composite coating, Corrosion Science, 155 (2019) 186-194.
[28] I. A. W. Ma, A. Shafaamri, R. Kasi, F. N. Zaini, V. Balakrishnan, R. Subramaniam, A. K. Arof, Anticorrosion properties of epoxy/nanocellulose nanocomposite coating, Bioresources, 12 (2017) 2912-2929.
[29] Y. He, Y. Boluk, J. Pan, A. Ahniyaz, T. Deltin, P. M. Claesson, Comparative study of CNC and CNF as additives in waterborne acrylate-based anti-corrosion coatings, Journal of Dispersion Science and Technology, 41 (2020) 2037-2047.
[30] M. Abd EI-Fattah, A. M. Hasan, M. Keshawy, A. M. El Saeed, O. M. Aboelenien, Nanocrystalline cellulose as an eco-friendly reinforcing additive to polyurethane coating for augmented anticorrosive behavior, Carbohydrate polymers, 183 (2018) 311-318.
[31] Q. Chen, U. P. de Larraya, N. Garmendia, M. Lasheras-Zubiate, L. Cordero-Arias, S. Virtanen, A. R. Boccaccini, Electrophoretic deposition of cellulose nanocrystals (CNs) and CNs/alginate nanocomposite coatings and free standing membranes, Colloids and Surfaces B: Biointerfaces, 118 (2014) 41-48.
[32] A. Z. Siavashani, J. Mohammadi, M. Rottmar, B. Senturk, J. Nourmohammadi, B. Sadeghi, L. Huber, K. Maniura-Weber, Silk fibroin/sericin 3D sponges: the effect of sericin on structural and biological properties of fibroin, International journal of biological macromolecules, 153 (2020) 317-326.
[33] S. Ghalei, J. Nourmohammadi, A. Solouk, H. Mirzadeh, Enhanced cellular response elicited by addition of amniotic fluid to alginate hydrogel-electrospun silk fibroin fibers for potential wound dressing application, Colloids and Surfaces B: Biointerfaces, 172 (2018) 82-89.
[34] F. Mottaghitalab, H. Hosseinkhani, M. A. Shokrgozar, C. Mao, M. Yang, M. Farokhi, Silk as a potential candidate for bone tissue engineering, Journal of controlled release, 215 (2015) 112-128.
[35] A. E. Thurber, F. G. Omenetto, D. L. Kaplan, In vivo bioresponses to silk proteins, Biomaterials, 71 (2015) 145-157.
[36] S. Zhang, X. Zhang, C. Zhao, J. Li, Y. Song, C. Xie, H. Tao, Y. Zhang, Y. He, Y. Jiang, Research on an Mg—Zn alloy as a degradable biomaterial, Acta biomaterialia, 6 (2010) 626-640.
[37] X. He, G. Zhang, Y. Pei, H. Zhang, Layered hydroxide/polydopamine/hyaluronic acid functionalized magnesium alloys for enhanced anticorrosion, biocompatibility and antithrombogenicity in vascular stents, Journal of biomaterials applications, 34 (2020) 1131-1141.
[38] S. Ghalei, A. Mondal, S. Hopkins, P. Singha, R. Devine, H. Handa, Silk Nanoparticles: A Natural Polymeric Platform for Nitric Oxide Delivery in Biomedical Applications, ACS Applied Materials & Interfaces, 12 (2020) 53615-53623.
[39] F. Singer, M. Schlesak, C. Mebert, S. Höhn, S. Virtanen, Corrosion properties of polydopamine coatings formed in one-step immersion process on magnesium, ACS applied materials & interfaces, 7 (2015) 26758-26766.
[40] A. Chakrabarty, Y. Teramoto, Recent advances in nanocellulose composites with polymers: a guide for choosing partners and how to incorporate them, Polymers, 10 (2018) 517.
[41] C. Wang, H. Fang, C. Hang, Y. Sun, Z. Peng, W. Wei, Y. Wang, Fabrication and characterization of silk fibroin coating on APTES pretreated Mg—Zn—Ca alloy, Materials Science and Engineering: C, 110 (2020) 110742.
[42] S. Ghalei, H. Asadi, B. Ghalei, Zein nanoparticle-embedded electrospun PVA nanofibers as wound dressing for topical delivery of anti-inflammatory diclofenac, Journal of Applied Polymer Science, 135 (2018) 46643.
[43] X. Hu, D. Kaplan, P. Cebe, Determining beta-sheet crystallinity in fibrous proteins by thermal analysis and infrared spectroscopy, Macromolecules, 39 (2006) 6161-6170.
[44] P. Wang, P. Xiong, J. Liu, S. Gao, T. Xi, Y. Cheng, A silk-based coating containing GREDVY peptide and heparin on Mg—Zn—Y—Nd alloy: improved corrosion resistance, hemocompatibility and endothelialization, Journal of Materials Chemistry B, 6 (2018) 966-978.
[45] S. Ghalei, S. Hopkins, M. Douglass, M. Garren, A. Mondal, H. Handa, Nitric oxide releasing halloysite nanotubes for biomedical applications, Journal of Colloid and Interface Science, (2021).
[46] H. Mao, C. Wei, Y. Gong, S. Wang, W. Ding, Mechanical and water-resistant properties of eco-friendly chitosan membrane reinforced with cellulose nanocrystals, Polymers, 11 (2019) 166.
[47] C.-J. Pan, L.-Q. Pang, Y. Hou, Y.-B. Lin, T. Gong, T. Liu, W. Ye, H.-Y. Ding, Improving corrosion resistance and biocompatibility of magnesium alloy by sodium hydroxide and hydrofluoric acid treatments, Applied Sciences, 7 (2017) 33.
[48] L. Zhang, E. A. Mohammed, A. Adriaens, Synthesis and electrochemical behavior of a magnesium fluoride-polydopamine-stearic acid composite coating on AZ31 magnesium alloy, Surface and Coatings Technology, 307 (2016) 56-64.
[49] X. Yang, L. Duan, X. Ran, Effect of polydopamine coating on improving photostability of polyphenylene sulfide fiber, Polymer Bulletin, 74 (2017) 641-656.
[50] S. Chakravarty, N. Bhardwaj, B. B. Mandal, N. S. Sarma, Silk fibroin-carbon nanoparticle composite scaffolds: a cost effective supramolecular ‘turn off’chemiresistor for nitroaromatic explosive vapours, Journal of Materials Chemistry C, 4 (2016) 8920-8929.
[51] H. Asadi, A. Ghaee, J. Nourmohammadi, A. Mashak, Electrospun zein/graphene oxide nanosheet composite nanofibers with controlled drug release as antibacterial wound dressing, International Journal of Polymeric Materials and Polymeric Biomaterials, 69 (2020) 173-185.
[52] L. Liu, X. Yang, H. Yu, C. Ma, J. Yao, Biomimicking the structure of silk fibers via cellulose nanocrystal as β-sheet crystallite, RSC Advances, 4 (2014) 14304-14313.
[53] P. Dorishetty, R. Balu, S. S. Athukoralalage, T. L. Greaves, J. Mata, L. De Campo, N. Saha, A. C. Zannettino, N. K. Dutta, N. R. Choudhury, Tunable biomimetic hydrogels from silk fibroin and nanocellulose, ACS Sustainable Chemistry & Engineering, 8 (2020) 2375-2389.
[54] H. J. Kim, Y. J. Yang, H. J. Oh, S. Kimura, M. Wada, U.-J. Kim, Cellulose-silk fibroin hydrogels prepared in a lithium bromide aqueous solution, Cellulose, 24 (2017) 5079-5088.
[55] H. Fang, C. Wang, S. Zhou, Z. Zheng, T. Lu, G. Li, Y. Tian, T. Suga, Enhanced adhesion and anticorrosion of silk fibroin coated biodegradable Mg—Zn—Ca alloy via a two-step plasma activation, Corrosion Science, 168 (2020) 108466.
[56] S. Agarwal, M. Morshed, M.-N. Labour, D. Hoey, B. Duffy, J. Curtin, S. Jaiswal, Enhanced corrosion protection and biocompatibility of a PLGA-silane coating on AZ31 Mg alloy for orthopaedic applications, RSC advances, 6 (2016) 113871-113883.
[57] H.-J. Jin, J. Park, R. Valluzzi, P. Cebe, D. L. Kaplan, Biomaterial films of Bombyx M on silk Fibroin with Poly (ethylene oxide), Biomacromolecules, 5 (2004) 711-717.
[58] S. Hofmann, C. W. P. Foo, F. Rossetti, M. Textor, G. Vunjak-Novakovic, D. Kaplan, H. Merkle, L. Meinel, Silk fibroin as an organic polymer for controlled drug delivery, Journal of Controlled Release, 111 (2006) 219-227.
[59] H. M. Wong, Y. Zhao, F. K. Leung, T. Xi, Z. Zhang, Y. Zheng, S. Wu, K. D. Luk, K. M. Cheung, P. K. Chu, Functionalized polymeric membrane with enhanced mechanical and biological properties to control the degradation of magnesium alloy, Advanced healthcare materials, 6 (2017) 1601269.
[60] H. Lee, S. M. Dellatore, W. M. Miller, P. B. Messersmith, Mussel-inspired surface chemistry for multifunctional coatings, science, 318 (2007) 426-430.
[61] J. B. Choi, Y. S. Jang, S. Mi Byeon, J. Hwa Jang, Y. K. Kim, T. S. Bae, M. H. Lee, Effect of composite coating with poly-dopamine/PCL on the corrosion resistance of magnesium, International Journal of Polymeric Materials and Polymeric Biomaterials, 68 (2019) 328-337.
[62] N. Eliaz, Corrosion of metallic biomaterials: A review, Materials, 12 (2019) 407.
[63] V. Shkirskiy, A. D. King, O. Gharbi, P. Volovitch, J. R. Scully, K. Ogle, N. Birbilis, Revisiting the electrochemical impedance spectroscopy of magnesium with online inductively coupled plasma atomic emission spectroscopy, ChemPhysChem, 16 (2015) 536-539.
[64] H. Yu, G. Xu, X. Shen, X. Yan, C. Hu, Y. Wang, Corrosion resistance and infrared emissivity properties of EPDM (EPDM-g-MAH) film on low infrared emissivity PU/Cu coating, Electrochimica acta, 55 (2010) 1843-1847.
[65] C.-W. Yang, G.-K. Wang, Effect of Hydrothermal (Sr)-Hydroxyapatite Coatings on the Corrosion Resistance and Mg2+ Ion Release to Enhance Osteoblastic Cell Responses of AZ91 D Alloy, Materials, 13 (2020) 591.
[66] C.-J. Pan, Y. Hou, Y.-N. Wang, F. Gao, T. Liu, Y.-H. Hou, Y.-F. Zhu, W. Ye, L.-R. Wang, Effects of self-assembly of 3-phosphonopropionic acid, 3-aminopropyltrimethoxysilane and dopamine on the corrosion behaviors and biocompatibility of a magnesium alloy, Materials Science and Engineering: C, 67 (2016) 132-143.
[67] Y. Huang, L. Deng, P. Ju, L. Huang, H. Qian, D. Zhang, X. Li, H. A. Terryn, J. M. Mol, Triple-action self-healing protective coatings based on shape memory polymers containing dual-function microspheres, ACS applied materials & interfaces, 10 (2018) 23369-23379.
[68] B. Mingo, Y. Guo, R. Leiva-Garcia, B. Connolly, A. Matthews, A. Yerokhin, Smart Functionalization of Ceramic-coated AZ31 Magnesium Alloy, ACS Applied Materials & Interfaces, (2020).
[69] A. Carangelo, A. Acquesta, T. Monetta, In-vitro corrosion of AZ31 magnesium alloys by using a polydopamine coating, Bioactive materials, 4 (2019) 71-78.
[70] R. Arrabal, A. Pardo, M. Merino, M. Mohedano, P. CasajGs, K. Paucar, G. Garces, Effect of Nd on the corrosion behaviour of AM50 and AZ91 D magnesium alloys in 3.5 wt. % NaCl solution, Corrosion Science, 55 (2012) 301-312.
[71] C. Wang, J. Shen, F. Xie, B. Duan, X. Xie, A versatile dopamine-induced intermediate layer for polyether imides (PEI) deposition on magnesium to render robust and high inhibition performance, Corrosion Science, 122 (2017) 32-40.
[72] T. Monetta, A. Acquesta, A. Carangelo, N. Donato, F. Bellucci, Durability of AZ31 magnesium biodegradable alloys polydopamine aided: Part 1, Journal of magnesium and alloys, 5 (2017) 412-422.
[73] X. Li, Z. Weng, W. Yuan, X. Luo, H. M. Wong, X. Liu, S. Wu, K. Yeung, Y. Zheng, P. K. Chu, Corrosion resistance of dicalcium phosphate dihydrate/poly (lactic-co-glycolic acid) hybrid coating on AZ31 magnesium alloy, Corrosion Science, 102 (2016) 209-221.
[74] S. Feliu, Electrochemical Impedance Spectroscopy for the Measurement of the Corrosion Rate of Magnesium Alloys: Brief Review and Challenges, Metals, 10 (2020) 775.
[75] E. Ghali, W. Dietzel, K.-U. Kainer, General and localized corrosion of magnesium alloys: a critical review, Journal of materials engineering and performance, 13 (2004) 7-23.
[76] S. Abbaspoor, A. Ashrafi, R. Abolfarsi, Development of self-healing coatings based on ethyl cellulose micro/nano-capsules, Surface Engineering, 35 (2019) 273-280.
[77] B. Ramezanzadeh, S. Niroumandrad, A. Ahmadi, M. Mahdavian, M. M. Moghadam, Enhancement of barrier and corrosion protection performance of an epoxy coating through wet transfer of amino functionalized graphene oxide, Corrosion Science, 103 (2016) 283-304.
[78] Q. Lu, X. Hu, X. Wang, J. A. Kluge, S. Lu, P. Cebe, D. L. Kaplan, Water-insoluble silk films with silk I structure, Acta biomaterialia, 6 (2010) 1380-1387.
[79] S. Ghalei, J. Li, M. Douglass, M. Garren, H. Handa, Synergistic Approach to Develop Antibacterial Electrospun Scaffolds Using Honey and S-Nitroso-N-acetyl Penicillamine, ACS Biomaterials Science & Engineering.
[80] H. Bakhsheshi-Rad, E. Hamzah, A. Ismail, M. Aziz, M. Daroonparvar, E. Saebnoori, A. Chami, In vitro degradation behavior, antibacterial activity and cytotoxicity of TiO2-MAO/ZnHA composite coating on Mg alloy for orthopedic implants, Surface and Coatings Technology, 334 (2018) 450-460.
[81] P. Xiong, Z. Jia, W. Zhou, J. Yan, P. Wang, W. Yuan, Y. Li, Y. Cheng, Z. Guan, Y. Zheng, Osteogenic and pH stimuli-responsive self-healing coating on biomedical Mg-1Ca alloy, Acta biomaterialia, 92 (2019) 336-350.
[82] H. Maleki, M.-A. Shahbazi, S. Montes, S. H. Hosseini, M. R. Eskandari, S. Zaunschirm, T. Verwanger, S. Mathur, B. Milow, B. Krammer, Mechanically strong silica-silk fibroin bioaerogel: a hybrid scaffold with ordered honeycomb micromorphology and multiscale porosity for bone regeneration, ACS applied materials & interfaces, 11 (2019) 17256-17269.
[83] Qian, H., et al., Dual-action smart coatings with a self-healing superhydrophobic surface and anti-corrosion properties. 2017. 5(5): p. 2355-2364.
[84] Fang, H., et al., Exploration of the enhanced performances for silk fibroin/sodium alginate composite coatings on biodegradable Mg— Zn— Ca alloy. Journal of Magnesium and Alloys, 2020.
[85] Zomorodian, A., et al., Corrosion resistance of a composite polymeric coating applied on biodegradable AZ31 magnesium alloy. Acta Biomaterialia, 2013. 9(10): p. 8660-8670.

What is claimed is:

1. A biodegradable orthopedic implant, comprising:
an implant comprising a Mg alloy; and
a coating comprising silk fibroin.

2. The biodegradable orthopedic implant of claim 1, wherein the coating comprises a first layer, and a second layer;
wherein the first layer comprises polydopamine; and
wherein the second layer comprises silk fibroin.

3. The biodegradable orthopedic implant of claim 2, wherein the second layer further comprises a biocompatible polymer.

4. The biodegradable orthopedic implant of claim 3, wherein the biocompatible polymer is cellulose nanocrystal.

5. The biodegradable orthopedic implant of claim 3, wherein the biocompatible polymer and silk fibroin form a composite.

6. The biodegradable orthopedic implant of claim 1, wherein an oxide layer is disposed on an external surface of the implant.

7. The biodegradable orthopedic implant of claim 1, wherein the implant comprises a Mg—Al—Zn alloy.

8. The biodegradable orthopedic implant of claim 7, wherein the Mg—Al—Zn alloy has an Al content of about 3%.

9. The biodegradable orthopedic implant of claim 7, wherein the Mg—Al—Zn alloy is AZ31.

10. The biodegradable orthopedic implant of claim 4, wherein the second layer comprises about 0.5-3.0% (w/w) cellulose nanocrystal with respect to silk fibroin weight.

11. The biodegradable orthopedic implant of claim 3, wherein the first layer has sub-micrometer thickness, and wherein the second layer is from about, 8 μm to 14 μm thick or about 8.7 μm to 13.7 μm thick.

12. The biodegradable orthopedic implant of claim 6, wherein oxide layer is from about 2 μm to 20 μm thick.

13. A corrosion-resistant coating for orthopedic implants, comprising:
a first layer comprising polydopamine and a second layer comprising silk fibroin.

14. The corrosion-resistant coating of claim 13, wherein the second layer further comprises cellulose nanocrystals.

15. The corrosion-resistant coating of claim 13, wherein the coating is disposed on an orthopedic implant, the orthopedic implant having an oxide layer on an external surface.

16. The corrosion-resistant coating of claim 13, wherein the coating is disposed on an external surface of an orthopedic implant.

17. A method of making a coated orthopedic implant, comprising:
immersing a magnesium alloy implant in an alkaline solution to form an oxide-coated implant;
providing a layer comprising polydopamine on the oxide-coated implant; and
providing a coating comprising silk fibroin over the layer comprising polydopamine.

18. The method of claim 17, wherein the coating comprising silk fibroin further comprises cellulose nanocrystals.

19. The method of claim 17, further comprising applying a second coating comprising silk fibroin to the implant.

20. The method of claim 17, wherein the alkaline solution is about 0.1-5 M NaOH.

21. The method of claim 18, wherein the wherein the silk fibroin layer comprises about 0.5-3.0% (w/w) cellulose nanocrystal with respect to silk fibroin weight.
